SEQUENCE LISTING (2 sequences)

SEQ ID NO: 1 - DNA, 50 bp, Artificial Sequence
  16S_V3_F
  misc_feature (42): n is a, c, g or t
tcgtcggcagcgtcagatgtgtataagagacagcctacgggnggcwgcag

SEQ ID NO: 2 - DNA, 55 bp, Artificial Sequence
  16S_V4_R
gtctcgtgggctcggagatgtgtataagagacaggactachvgggtatctaatcc

(19) United States
(12) Patent Application Publication
(10) Pub. No.: US 2019/0330687 A1
(43) Pub. Date: Oct. 31, 2019

(54) METHOD FOR DIAGNOSING COLON TUMOR VIA BACTERIAL METAGENOMIC ANALYSIS

(71) Applicant: MD HEALTHCARE INC., Seoul (KR)

(72) Inventor: Yoon-Keun KIM, Namyangju-si, Gyeonggi-do (KR)

(73) Assignee: MD HEALTHCARE INC., Seoul (KR)

(21) Appl. No.: 16/472,986

(22) PCT Filed: Dec. 27, 2017
(86) PCT No.: PCT/KR2017/015557

§ 371 Date: Jun. 24, 2019

(30) Foreign Application Priority Data
Dec. 28, 2016 (KR) 10-2016-0181574
Dec. 26, 2017 (KR) 10-2017-0180144

(51) Int. Cl. C12Q 1/689; C12Q 1/6806

(52) U.S. Cl. CPC C12Q 1/689; C12Q 2600/112; C12Q 2531/113; C12Q 1/6806

(57) ABSTRACT

Provided are a method of predicting the onset and causative factors of colon tumors such as a colon polyp, colon cancer, and the like through metagenomic analysis of bacteria-derived extracellular vesicles present in a human body-derived substance, and a method of diagnosing causative factors and risk of colon tumors by sequencing of a metagenome present in bacteria-derived extracellular vesicles in stool or urine.

### TECHNICAL FIELD

The present invention relates to a method of diagnosing colon tumors through bacterial metagenomic analysis, and more particularly, to a method of diagnosing colon tumors such as colonic polyps, colon cancer, and the like by analyzing an increase or decrease in content of extracellular vesicles derived from specific bacteria through bacterial metagenomic analysis using a subject-derived sample.

### BACKGROUND ART

Colon cancer or colorectal cancer is a malignant tumor occurring in the appendix, colon, and rectum and occurs in the mucous membrane, which is the innermost surface of the large intestine. According to an announcement in 2006, colon cancer is the second most common cancer after gastric cancer in Korea, and the frequency of onset thereof has sharply increased as eating habits have recently been westernized, and in the last decade, mortality from colon cancer has increased by about 80% and the rate of increase is continuously increasing. Colon cancer occurs most frequently in those in their 60s, and as for an occurrence site, colon cancer occurs more frequently in the rectum than in the colon. Although colon cancer may occur at any age, 90% or more of patients with colon cancer are 40 years old or older, and the incidence rate has doubled every 10 years.

All colon cancers are known to begin with a colon polyp or colon adenoma, and a polyp refers to a protrusion occurring by initial abnormal growth of the epithelium in the intestinal lining, and is a very common disease occurring in 15% to 20% of adults. In addition to colon polyps, if you have a family member with colon cancer or you are an individual suffering from ulcerative colitis for a long period of time, the risk of colon cancer increases. In addition, colon cancer is a typical cancer known to be associated with foods, and as causative factors of colon cancer, low intake of cellulose due to westernization of dietary life, high intake of animal fats, excess intake of refined sugar, and the like are known.

In the case of early colon cancer, there is no specific symptom, but even if there is no symptom, unnoticed intestinal bleeding occurs and thus blood is lost, resulting in the occurrence of anemia, and sometimes, a lack of appetite and weight loss may occur. When cancer progresses, a stomachache or a change in bowel habits, such as diarrhea, constipation, or the like may occur, or there may be symptoms of rectal bleeding from the anus, and blood may have a bright scarlet color or a black color. When colon cancer is developed, a mass that has not been usually recognized in the abdomen may be felt. The most notable symptoms are a change in bowel habits, bloody stool, pain, and anemia, and particularly, it is necessary to thoroughly investigate such changes in adults aged 40 or more years old.

The definite diagnosis of colon cancer is possible when cancer cells are detected through a biopsy by colonoscopy. In most colon cancer cases, there is no early symptom so that diagnosis is very difficult, and there is currently no method of predicting colon cancer using a non-invasive method. There are many cases in which solid cancers such as colon cancer and the like are detected using an existing diagnosis method after cancer development, and thus to reduce medical costs and prevent death due to colon cancer, it is effective to provide a method of preventing the onset of colon cancer in a high risk group by predicting the occurrence of colon tumor and causative factors.

Meanwhile, it is known that the number of microorganisms symbiotically living in the human body is 100 trillion which is 10 times the number of human cells, and the number of genes of microorganisms exceeds 100 times the number of human genes. A microbiota or microbiome is a microbial community that includes bacteria, archaea, and eukaryotes present in a given habitat. The intestinal microbiota is known to play a vital role in human's physiological phenomena and significantly affect human health and diseases through interactions with human cells. Bacteria coexisting in human bodies secrete nanometer-sized vesicles to exchange information about genes, proteins, and the like with other cells. The mucous membranes form a physical barrier membrane that does not allow particles with the size of 200 nm or more to pass therethrough, and thus bacteria symbiotically living in the mucous membranes are unable to pass therethrough, but bacteria-derived extracellular vesicles have a size of approximately 100 nm or less and thus relatively freely pass through the mucous membranes and are absorbed into the human body.

Metagenomics, also called environmental genomics, may be analytics for metagenomic data obtained from samples collected from the environment (Korean Patent Publication No. 2011-0073049). Recently, the bacterial composition of human microbiota has been listed using a method based on 16s ribosomal RNA (16s rRNA) base sequences, and 16s rDNA base sequences, which are genes of 16s ribosomal RNA, are analyzed using a next generation sequencing (NGS) platform. However, in the onset of colon tumors, there has been no report of a method of diagnosing colon tumors by isolating bacteria and bacteria-derived vesicles from a human-derived substance such as stool and identifying causative factors of colon tumors such as colon polyps, colon cancer, and the like through analysis of metagenomes present in the bacteria-derived vesicles.

### DISCLOSURE

### Technical Problem

To diagnose colon tumors such as colon polyps, colon cancer, and the like, the inventors of the present invention extracted DNA from bacteria-derived extracellular vesicles using urine and stool, which is a subject-derived sample, and performed metagenomic analysis on the extracted DNA, and, as a result, identified bacteria-derived extracellular vesicles capable of acting as a causative factor of colon tumors such as colon polyps, colon cancer, and the like, thus completing the present invention based on these findings.

Therefore, it is an object of the present invention to provide a method of providing information for colon tumor diagnosis by metagenomic analysis of genes present in bacteria-derived extracellular vesicles.

However, the technical goals of the present invention are not limited to the aforementioned goals, and other unmentioned technical goals will be clearly understood by those of ordinary skill in the art from the following description.

### Technical Solution

To achieve the above-described object of the present invention, the present invention provides a method of providing information for colon tumor diagnosis, comprising the following processes:

(a) extracting DNA from extracellular vesicles isolated from a subject sample;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles between a normal individual-derived sample and a colon cancer patient-derived sample through sequencing of a product of the PCR,

comparing an increase or decrease in content of bacteria-derived extracellular vesicles between a colon polyp patient-derived sample and a colon cancer patient-derived sample through sequencing of a product of the PCR, or

comparing an increase or decrease in content of bacteria-derived extracellular vesicles between a normal individual-derived sample and a colon polyp patient-derived sample through sequencing of a product of the PCR.

The present invention also provides a method of diagnosing colon tumor, comprising the following processes:

(a) extracting DNA from extracellular vesicles isolated from a subject sample;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles between a normal individual-derived sample and a colon cancer patient-derived sample through sequencing of a product of the PCR,

comparing an increase or decrease in content of bacteria-derived extracellular vesicles between a colon polyp patient-derived sample and a colon cancer patient-derived sample through sequencing of a product of the PCR, or

comparing an increase or decrease in content of bacteria-derived extracellular vesicles between a normal individual-derived sample and a colon polyp patient-derived sample through sequencing of a product of the PCR.

The present invention also provides a method of predicting a risk for colon tumor, comprising the following processes:

(a) extracting DNA from extracellular vesicles isolated from a subject sample;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles between a normal individual-derived sample and a colon cancer patient-derived sample through sequencing of a product of the PCR,

comparing an increase or decrease in content of bacteria-derived extracellular vesicles between a colon polyp patient-derived sample and a colon cancer patient-derived sample through sequencing of a product of the PCR, or

comparing an increase or decrease in content of bacteria-derived extracellular vesicles between a normal individual-derived sample and a colon polyp patient-derived sample through sequencing of a product of the PCR.

In one embodiment of the present invention, in process (c), colon cancer may be diagnosed by comparing an increase or decrease in content of bacteria-derived extracellular vesicles between a normal individual-derived sample and a colon cancer patient-derived sample through sequencing of a product of the PCR.

In one embodiment of the present invention, in process (c), the comparing may comprise comparing an increase or decrease in content of:

extracellular vesicles derived from one or more bacteria selected from the group consisting of the phylum Deferribacteres, the phylum Tenericutes, the phylum Actinobacteria, the phylum Acidobacteria, the phylum Armatimonadetes, the phylum Planctomycetes, the phylum Fusobacteria, the phylum Proteobacteria, and the phylum Euryarchaeota;

extracellular vesicles derived from one or more bacteria selected from the group consisting of the class Deferribacteres, the class Mollicutes, the class 4C0d-2, the class Bacilli, the class Alphaproteobacteria, the class Saprospirae, the class Fimbriimonadia, the class Acidobacteria-6, the class Solibacteres, the class Coriobacteriia, the class Oscillatoriophycideae, the class Fusobacteriia, the class Gammaproteobacteria, the class Clostridia, and the class Methanobacteria;

extracellular vesicles derived from one or more bacteria selected from the group consisting of the order RF32, the order YS2, the order Deferribacterales, the order Turicibacterales, the order RF39, the order Oceanospirillales, the order Rhizobiales, the order Lactobacillales, the order Rhodobacterales, the order Saprospirales, the order Sphingomonadales, the order Fimbriimonadales, the order iii1-15, the order Solibacterales, the order Coriobacteriales, the order Chroococcales, the order Fusobacteriales, the order Bdellovibrionales, the order Desulfobacterales, the order Stramenopiles, the order Pseudomonadales, the order Clostridiales, the order Desulfovibrionales, and the order Methanobacteriales;

extracellular vesicles derived from one or more bacteria selected from the group consisting of the family Peptococcaceae, the family Deferribacteraceae, the family Turicibacteraceae, the family Halomonadaceae, the family Clostridiaceae, the family Prevotellaceae, the family Peptostreptococcaceae, the family Rhodobacteraceae, the family Nocardioidaceae, the family Sphingomonadaceae, the family Bartonellaceae, the family Cellulomonadaceae, the family Lactobacillaceae, the family Rhizobiaceae, the family Fimbriimonadaceae, the family Dermacoccaceae, the family Leptotrichiaceae, the family Coriobacteriaceae, the family Xenococcaceae, the family Aeromonadaceae, the family Geodermatophilaceae, the family Bdellovibrionaceae, the family Moraxellaceae, the family Pseudomonadaceae, the family Streptococcaceae, the family Veillonellaceae, the family Bacteroidaceae, the family Aerococcaceae, the family Comamonadaceae, the family Paraprevotellaceae, the family Christensenellaceae, the family Ruminococcaceae, the family Corynebacteriaceae, the family Gordoniaceae, the family Mycobacteriaceae, the family Desulfovibrionaceae, the family Alcaligenaceae, the family Barnesiellaceae, the family Methanobacteriaceae, and the family Rikenellaceae; or

extracellular vesicles derived from one or more bacteria selected from the group consisting of the genus rc4-4, the genus Proteus, the genus Catenibacterium, the genus Mucispirillum, the genus Eubacterium, the genus Turicibacter, the genus Alloiococcus, the genus Halomonas, the genus Prevotella, the genus Dialister, the genus Anaerostipes, the genus SMB53, the genus Faecalibacterium, the genus Blautia, the genus Capnocytophaga, the genus Sphingomonas, the genus Lactobacillus, the genus Fimbriimonas, the genus Dermacoccus, the genus Achromobacter, the genus Novosphingobium, the genus Sneathia, the genus Agrobacterium, the genus Blastomonas, the genus Bdellovibrio, the genus Alkanindiges, the genus Roseateles, the genus Shuttleworthia, the genus Rhizobium, the genus Morganella, the genus Acinetobacter, the genus Pseudomonas, the genus Enterococcus, the genus Lactococcus, the genus Coprococcus, the genus Bacteroides, the genus Dorea, the genus Streptococcus, the genus Lachnospira, the genus Ruminococcus, the genus Corynebacterium, the genus Comamonas, the genus Gordonia, the genus Paraprevotella, the genus Mycobacterium, the genus Roseburia, the genus Slackia, the genus Escherichia, the genus Phascolarctobacterium, the genus Sutterella, the genus Virgibacillus, the genus Eggerthella, the genus Citrobacter, the genus Roseomonas, the genus Serratia, the genus Methanobrevibacter, and the genus Bilophila.

In one embodiment of the present invention, in process (c), colon cancer may be diagnosed by comparing an increase or decrease in content of bacteria-derived extracellular vesicles between a colon polyp patient-derived sample and a colon cancer patient-derived sample through sequencing of a product of the PCR.

In one embodiment of the present invention, in process (c), the comparing may comprise comparing an increase or decrease in content of:

extracellular vesicles derived from the bacteria belonging to the phylum Spirochaetes;

extracellular vesicles derived from one or more bacteria selected from the group consisting of the class Spirochaetes and the class Acidobacteria-6;

extracellular vesicles derived from one or more bacteria selected from the group consisting of the order Spirochaetales and the order Myxococcales;

extracellular vesicles derived from one or more bacteria selected from the group consisting of the family Spirochaetaceae and the family S24-7; or

extracellular vesicles derived from one or more bacteria selected from the group consisting of the genus Treponema, the genus Dialister, the genus Oscillospira, and the genus Eubacterium.

In one embodiment of the present invention, in process (c), colon polyp may be diagnosed by comparing an increase or decrease in content of bacteria-derived extracellular vesicles between a normal individual-derived sample and a colon polyp patient-derived sample through sequencing of a product of the PCR.

In one embodiment of the present invention, in process (c), the comparing may comprise comparing an increase or decrease in content of:

extracellular vesicles derived from one or more bacteria selected from the group consisting of the phylum Actinobacteria, the phylum Proteobacteria, and the phylum Euryarchaeota;

extracellular vesicles derived from one or more bacteria selected from the group consisting of the class Betaproteobacteria, the class Solibacteres, the class Gammaproteobacteria, the class Clostridia, the class Methanobacteria, and the class 4C0d-2;

extracellular vesicles derived from one or more bacteria selected from the group consisting of the order Burkholderiales, the order Sphingomonadales, the order Solibacterales, the order Stramenopiles, the order Pseudomonadales, the order Clostridiales, the order Oceanospirillales, the order Desulfovibrionales, and the order Methanobacteriales;

extracellular vesicles derived from one or more bacteria selected from the group consisting of the family Rhizobiaceae, the family Sphingomonadaceae, the family Exiguobacteraceae, the family Moraxellaceae, the family Pseudomonadaceae, the family Streptococcaceae, the family Peptostreptococcaceae, the family Comamonadaceae, the family Veillonellaceae, the family Bacteroidaceae, the family Paraprevotellaceae, the family Ruminococcaceae, the family Corynebacteriaceae, the family Christensenellaceae, the family Odoribacteraceae, the family Desulfovibrionaceae, the family Halomonadaceae, the family Alcaligenaceae, the family Barnesiellaceae, the family Methanobacteriaceae, and the family Rikenellaceae; or

extracellular vesicles derived from one or more bacteria selected from the group consisting of the genus Sphingomonas, the genus Alkanindiges, the genus Roseateles, the genus Rhizobium, the genus Morganella, the genus Proteus, the genus Exiguobacterium, the genus Acinetobacter, the genus Pseudomonas, the genus SMB53, the genus Lactococcus, the genus Coprococcus, the genus Streptococcus, the genus Bacteroides, the genus Ruminococcus, the genus Corynebacterium, the genus Odoribacter, the genus Clostridium, the genus Comamonas, the genus Paraprevotella, the genus Roseburia, the genus Citrobacter, the genus Klebsiella, the genus Virgibacillus, the genus Slackia, the genus Dialister, the genus Phascolarctobacterium, the genus Sutterella, the genus Halomonas, the genus Roseomonas, and the genus Methanobrevibacter.

In one embodiment of the present invention, the subject sample may be stool or urine.

### Advantageous Effects

Extracellular vesicles secreted from bacteria present in the environment are absorbed into the human body, and thus may directly affect the occurrence of inflammation and cancer, and it is difficult to diagnose colon polyp and colon cancer early before symptoms occur, and thus efficient treatment therefor is difficult. Thus, according to the present invention, a risk of developing colon tumors such as colon polyps, colon cancer, and the like can be predicted through metagenomic analysis of bacteria or bacteria-derived extracellular vesicles by using a human body-derived sample, and thus the onset of colon tumor can be delayed or colon tumor can be prevented through appropriate management by early diagnosis and prediction of a risk group for colon tumor, and, even after colon tumor occur, early diagnosis for colon tumor can be implemented, thereby lowering the incidence rate of colon tumor and increasing therapeutic effects. In addition, patients diagnosed with a colon polyp or colon cancer can avoid exposure to causative factors predicted by metagenomic analysis, whereby the progression of a colon polyp and colon cancer can be ameliorated, or recurrence thereof can be prevented.

### DESCRIPTION OF DRAWINGS

FIGS. 1A and 1B are views for evaluating the distribution pattern of extracellular vesicles (EVs) derived from bacteria in vivo. FIG. 1A illustrates images showing the distribution pattern of intestinal bacteria and EVs derived from bacteria per time (0 h, 5 min, 3 h, 6 h, and 12 h) after being orally administered to mice. FIG. 1B illustrates images showing the distribution pattern of gut bacteria and EVs derived from bacteria after being orally administered to mice and, after 12 hours, blood and various organs (heart, lung, liver, kidney, spleen, adipose tissue, and muscle) of the mice were extracted.

FIG. 2 illustrates distribution results of bacteria-derived EVs exhibiting significant diagnostic performance at a phylum level, after metagenomic analysis of bacteria-derived EVs isolated from colon cancer patient-derived stool and normal individual-derived stool.

FIG. 3 illustrates distribution results of bacteria-derived EVs exhibiting significant diagnostic performance at a class level, after metagenomic analysis of bacteria-derived EVs isolated from colon cancer patient-derived stool and normal individual-derived stool.

FIG. 4 illustrates distribution results of bacteria-derived EVs exhibiting significant diagnostic performance at an order level, after metagenomic analysis of bacteria-derived EVs isolated from colon cancer patient-derived stool and normal individual-derived stool.

FIG. 5 illustrates distribution results of bacteria-derived EVs exhibiting significant diagnostic performance at a family level, after metagenomic analysis of bacteria-derived EVs isolated from colon cancer patient-derived stool and normal individual-derived stool.

FIG. 6 illustrates distribution results of bacteria-derived EVs exhibiting significant diagnostic performance at a genus level, after metagenomic analysis of bacteria-derived EVs isolated from colon cancer patient-derived stool and normal individual-derived stool.

FIG. 7 illustrates distribution results of bacteria-derived EVs exhibiting significant diagnostic performance at a phylum level, after metagenomic analysis of bacteria-derived EVs isolated from colon cancer patient-derived urine and normal individual-derived urine.

FIG. 8 illustrates distribution results of bacteria-derived EVs exhibiting significant diagnostic performance at a class level, after metagenomic analysis of bacteria-derived EVs isolated from colon cancer patient-derived urine and normal individual-derived urine.

FIG. 9 illustrates distribution results of bacteria-derived EVs exhibiting significant diagnostic performance at an order level, after metagenomic analysis of bacteria-derived EVs isolated from colon cancer patient-derived urine and normal individual-derived urine.

FIG. 10 illustrates distribution results of bacteria-derived EVs exhibiting significant diagnostic performance at a family level, after metagenomic analysis of bacteria-derived EVs isolated from colon cancer patient-derived urine and normal individual-derived urine.

FIG. 11 illustrates distribution results of bacteria-derived EVs exhibiting significant diagnostic performance at a genus level, after metagenomic analysis of bacteria-derived EVs isolated from colon cancer patient-derived urine and normal individual-derived urine.

FIG. 12 illustrates distribution results of bacteria-derived EVs exhibiting significant diagnostic performance at a phylum level, after metagenomic analysis of bacteria-derived EVs isolated from colon cancer patient-derived stool and colon polyp patient-derived stool.

FIG. 13 illustrates distribution results of bacteria-derived EVs exhibiting significant diagnostic performance at a class level, after metagenomic analysis of bacteria-derived EVs isolated from colon cancer patient-derived stool and colon polyp patient-derived stool.

FIG. 14 illustrates distribution results of bacteria-derived EVs exhibiting significant diagnostic performance at an order level, after metagenomic analysis of bacteria-derived EVs isolated from colon cancer patient-derived stool and colon polyp patient-derived stool.

FIG. 15 illustrates distribution results of bacteria-derived EVs exhibiting significant diagnostic performance at a family level, after metagenomic analysis of bacteria-derived EVs isolated from colon cancer patient-derived stool and colon polyp patient-derived stool.

FIG. 16 illustrates distribution results of bacteria-derived EVs exhibiting significant diagnostic performance at a genus level, after metagenomic analysis of bacteria-derived EVs isolated from colon cancer patient-derived stool and colon polyp patient-derived stool.

FIG. 17 illustrates distribution results of bacteria-derived EVs exhibiting significant diagnostic performance at an order level, after metagenomic analysis of bacteria-derived EVs isolated from colon cancer patient-derived urine and colon polyp patient-derived urine.

FIG. 18 illustrates distribution results of bacteria-derived EVs exhibiting significant diagnostic performance at a genus level, after metagenomic analysis of bacteria-derived EVs isolated from colon cancer patient-derived urine and colon polyp patient-derived urine.

FIG. 19 illustrates distribution results of bacteria-derived EVs exhibiting significant diagnostic performance at a phylum level, after metagenomic analysis of bacteria-derived EVs isolated from colon polyp patient-derived stool and normal individual-derived stool.

FIG. 20 illustrates distribution results of bacteria-derived EVs exhibiting significant diagnostic performance at a class level, after metagenomic analysis of bacteria-derived EVs isolated from colon polyp patient-derived stool and normal individual-derived stool.

FIG. 21 illustrates distribution results of bacteria-derived EVs exhibiting significant diagnostic performance at an order level, after metagenomic analysis of bacteria-derived EVs isolated from colon polyp patient-derived stool and normal individual-derived stool.

FIG. 22 illustrates distribution results of bacteria-derived EVs exhibiting significant diagnostic performance at a family level, after metagenomic analysis of bacteria-derived EVs isolated from colon polyp patient-derived stool and normal individual-derived stool.

FIG. 23 illustrates distribution results of bacteria-derived EVs exhibiting significant diagnostic performance at a genus level, after metagenomic analysis of bacteria-derived EVs isolated from colon polyp patient-derived stool and normal individual-derived stool.

FIG. 24 illustrates distribution results of bacteria-derived EVs exhibiting significant diagnostic performance at a phylum level, after metagenomic analysis of bacteria-derived EVs isolated from colon polyp patient-derived urine and normal individual-derived urine.

FIG. 25 illustrates distribution results of bacteria-derived EVs exhibiting significant diagnostic performance at a class level, after metagenomic analysis of bacteria-derived EVs isolated from colon polyp patient-derived urine and normal individual-derived urine.

FIG. 26 illustrates distribution results of bacteria-derived EVs exhibiting significant diagnostic performance at an order level, after metagenomic analysis of bacteria-derived EVs isolated from colon polyp patient-derived urine and normal individual-derived urine.

FIG. 27 illustrates distribution results of bacteria-derived EVs exhibiting significant diagnostic performance at a family level, after metagenomic analysis of bacteria-derived EVs isolated from colon polyp patient-derived urine and normal individual-derived urine.

FIG. 28 illustrates distribution results of bacteria-derived EVs exhibiting significant diagnostic performance at a genus level, after metagenomic analysis of bacteria-derived EVs isolated from colon polyp patient-derived urine and normal individual-derived urine.

### BEST MODE

The present invention relates to a method of diagnosing colon tumors such as colon polyps, colon cancer, and the like through bacterial metagenomic analysis. The inventors of the present invention extracted genes from bacteria-derived extracellular vesicles present in subject-derived samples, performed metagenomic analysis thereon, and identified bacteria-derived extracellular vesicles capable of acting as a causative factor of colon tumors such as colon polyps, colon cancer, and the like.

Thus, the present invention provides a method of providing information for colon tumor diagnosis, comprising the following processes:

(a) extracting DNA from extracellular vesicles isolated from a subject sample;

(b) performing polymerase chain reaction (PCR) on the extracted DNA using a pair of primers having SEQ ID NO: 1 and SEQ ID NO: 2; and

(c) comparing an increase or decrease in content of bacteria-derived extracellular vesicles between a normal individual-derived sample and a colon cancer patient-derived sample through sequencing of a product of the PCR,

comparing an increase or decrease in content of bacteria-derived extracellular vesicles between a colon polyp patient-derived sample and a colon cancer patient-derived sample through sequencing of a product of the PCR, or

comparing an increase or decrease in content of bacteria-derived extracellular vesicles between a normal individual-derived sample and a colon polyp patient-derived sample through sequencing of a product of the PCR.

The term “colon cancer diagnosis” as used herein refers to determining whether a patient has a risk for colon cancer, whether the risk for colon cancer is relatively high, or whether colon cancer has already occurred. The method of the present invention may be used to delay the onset of colon cancer through special and appropriate care for a specific patient, which is a patient having a high risk for colon cancer or prevent the onset of colon cancer. In addition, the method may be clinically used to determine treatment by selecting the most appropriate treatment method through early diagnosis of colon cancer.

The term “colon polyp diagnosis” as used herein refers to determining whether a patient has a risk for colon polyp, whether the risk for colon polyp is relatively high, or whether colon polyp has already occurred. The method of the present invention may be used to delay the onset of colon polyp through special and appropriate care for a specific patient, which is a patient having a high risk for colon polyp or prevent the onset of colon polyp. In addition, the method may be clinically used to determine treatment by selecting the most appropriate treatment method through early diagnosis of colon polyp.

The term “metagenome” as used herein refers to the total of genomes including all viruses, bacteria, fungi, and the like in isolated regions such as soil, the intestines of animals, and the like, and is mainly used as a concept of genomes that explains identification of many microorganisms at once using a sequencer to analyze non-cultured microorganisms. In particular, a metagenome does not refer to a genome of one species, but refers to a mixture of genomes, including genomes of all species of an environmental unit. This term originates from the view that, when defining one species in a process in which biology is advanced into omics, various species as well as existing one species functionally interact with each other to form a complete species. Technically, it is the subject of techniques that analyzes all DNAs and RNAs regardless of species using rapid sequencing to identify all species in one environment and verify interactions and metabolism. In the present invention, bacterial metagenomic analysis is performed using bacteria-derived extracellular vesicles isolated from, for example, serum.

In an embodiment of the present invention, metagenomic analysis was performed on genes present in bacteria-derived extracellular vesicles in stool and urine samples of normal individuals, colon polyp patients, and colon cancer patients, and bacteria-derived extracellular vesicles capable of acting as causes of the onset of colon cancer and a colon polyp were actually identified by analysis at phylum, class, order, family, and genus levels.

More particularly, in one embodiment of the present invention, as a result of performing metagenomic analysis on bacteria-derived extracellular vesicles present in stool at a phylum level, the content of extracellular vesicles derived from bacteria belonging to the phylum Deferribacteres, the phylum Tenericutes, the phylum Actinobacteria, the phylum Acidobacteria, the phylum Armatimonadetes, the phylum Planctomycetes, and the phylum Fusobacteria was significantly different between colon cancer patients and normal individuals (see Example 4).

More particularly, in one embodiment of the present invention, as a result of performing metagenomic analysis on bacteria-derived extracellular vesicles present in stool at a class level, the content of extracellular vesicles derived from bacteria belonging to the class Deferribacteres, the class Mollicutes, the class 4C0d-2, the class Bacilli, the class Alphaproteobacteria, the class Saprospirae, the class Fimbriimonadia, the class Acidobacteria-6, the class Solibacteres, the class Coriobacteriia, the class Oscillatoriophycideae, and the class Fusobacteriia was significantly different between colon cancer patients and normal individuals (see Example 4).

More particularly, in one embodiment of the present invention, as a result of performing metagenomic analysis on bacteria-derived extracellular vesicles present in stool at an order level, the content of extracellular vesicles derived from bacteria belonging to the order RF32, the order YS2, the order Deferribacterales, the order Turicibacterales, the order RF39, the order Oceanospirillales, the order Rhizobiales, the order Lactobacillales, the order Rhodobacterales, the order Saprospirales, the order Sphingomonadales, the order Fimbriimonadales, the order iii1-15, the order Solibacterales, the order Coriobacteriales, the order Chroococcales, the order Fusobacteriales, and the order Bdellovibrionales was significantly different between colon cancer patients and normal individuals (see Example 4).

More particularly, in one embodiment of the present invention, as a result of performing metagenomic analysis on bacteria-derived extracellular vesicles present in stool at a family level, the content of extracellular vesicles derived from bacteria belonging to the family Peptococcaceae, the family Deferribacteraceae, the family Turicibacteraceae, the family Halomonadaceae, the family Clostridiaceae, the family Prevotellaceae, the family Peptostreptococcaceae, the family Rhodobacteraceae, the family Nocardioidaceae, the family Sphingomonadaceae, the family Bartonellaceae, the family Cellulomonadaceae, the family Lactobacillaceae, the family Rhizobiaceae, the family Fimbriimonadaceae, the family Dermacoccaceae, the family Leptotrichiaceae, the family Coriobacteriaceae, the family Xenococcaceae, the family Aeromonadaceae, the family Geodermatophilaceae, and the family Bdellovibrionaceae was significantly different between colon cancer patients and normal individuals (see Example 4).

More particularly, in one embodiment of the present invention, as a result of performing metagenomic analysis on bacteria-derived extracellular vesicles present in stool at a genus level, the content of extracellular vesicles derived from bacteria belonging to the genus rc4-4, the genus Proteus, the genus Catenibacterium, the genus Mucispirillum, the genus Eubacterium, the genus Turicibacter, the genus Alloiococcus, the genus Halomonas, the genus Prevotella, the genus Dialister, the genus Anaerostipes, the genus SMB53, the genus Faecalibacterium, the genus Blautia, the genus Capnocytophaga, the genus Sphingomonas, the genus Lactobacillus, the genus Fimbriimonas, the genus Dermacoccus, the genus Achromobacter, the genus Novosphingobium, the genus Sneathia, the genus Agrobacterium, the genus Blastomonas, the genus Bdellovibrio, the genus Alkanindiges, the genus Roseateles, and the genus Shuttleworthia was significantly different between colon cancer patients and normal individuals (see Example 4).

More particularly, in one embodiment of the present invention, as a result of performing metagenomic analysis on bacteria-derived extracellular vesicles present in urine at a phylum level, the content of extracellular vesicles derived from bacteria belonging to the phylum Proteobacteria and the phylum Euryarchaeota was significantly different between colon cancer patients and normal individuals (see Example 5).

More particularly, in one embodiment of the present invention, as a result of performing metagenomic analysis on bacteria-derived extracellular vesicles present in urine at a class level, the content of extracellular vesicles derived from bacteria belonging to the class Gammaproteobacteria, the class Clostridia, and the class Methanobacteria was significantly different between colon cancer patients and normal individuals (see Example 5).

More particularly, in one embodiment of the present invention, as a result of performing metagenomic analysis on bacteria-derived extracellular vesicles present in urine at an order level, the content of extracellular vesicles derived from bacteria belonging to the order Desulfobacterales, the order Stramenopiles, the order Pseudomonadales, the order Clostridiales, the order Turicibacterales, the order Desulfovibrionales, the order Oceanospirillales, and the order Methanobacteriales was significantly different between colon cancer patients and normal individuals (see Example 5).

More particularly, in one embodiment of the present invention, as a result of performing metagenomic analysis on bacteria-derived extracellular vesicles present in urine at a family level, the content of extracellular vesicles derived from bacteria belonging to the family Moraxellaceae, the family Pseudomonadaceae, the family Streptococcaceae, the family Turicibacteraceae, the family Veillonellaceae, the family Bacteroidaceae, the family Aerococcaceae, the family Comamonadaceae, the family Clostridiaceae, the family Paraprevotellaceae, the family Christensenellaceae, the family Ruminococcaceae, the family Corynebacteriaceae, the family Gordoniaceae, the family Mycobacteriaceae, the family Desulfovibrionaceae, the family Halomonadaceae, the family Alcaligenaceae, the family Barnesiellaceae, the family Methanobacteriaceae, and the family Rikenellaceaewas significantly different between colon cancer patients and normal individuals (see Example 5).

More particularly, in one embodiment of the present invention, as a result of performing metagenomic analysis on bacteria-derived extracellular vesicles present in urine at a genus level, the content of extracellular vesicles derived from bacteria belonging to the genus Rhizobium, the genus Proteus, the genus Morganella, the genus Acinetobacter, the genus Pseudomonas, the genus SMB53, the genus Enterococcus, the genus Lactococcus, the genus Turicibacter, the genus Coprococcus, the genus Bacteroides, the genus Dorea, the genus Streptococcus, the genus Lachnospira, the genus Ruminococcus, the genus Corynebacterium, the genus Comamonas, the genus Gordonia, the genus Paraprevotella, the genus Mycobacterium, the genus Roseburia, the genus Dialister, the genus Slackia, the genus Escherichia, the genus Phascolarctobacterium, the genus Sutterella, the genus Virgibacillus, the genus Eggerthella, the genus Halomonas, the genus Citrobacter, the genus Roseomonas, the genus Alloiococcus, the genus Serratia, the genus Methanobrevibacter, and the genus Bilophila significantly different between colon cancer patients and normal individuals (see Example 5).

More particularly, in one embodiment of the present invention, as a result of performing metagenomic analysis on bacteria-derived extracellular vesicles present in stool at a phylum level, the content of extracellular vesicles derived from bacteria belonging to the phylum Spirochaetes was significantly different between colon cancer patients and colon polyp patients (see Example 6).

More particularly, in one embodiment of the present invention, as a result of performing metagenomic analysis on bacteria-derived extracellular vesicles present in stool at a class level, the content of extracellular vesicles derived from bacteria belonging to the class Spirochaetes and the class Acidobacteria-6 was significantly different between colon cancer patients and colon polyp patients (see Example 6).

More particularly, in one embodiment of the present invention, as a result of performing metagenomic analysis on bacteria-derived extracellular vesicles present in stool at an order level, the content of extracellular vesicles derived from bacteria belonging to the order Spirochaetales was significantly different between colon cancer patients and colon polyp patients (see Example 6).

More particularly, in one embodiment of the present invention, as a result of performing metagenomic analysis on bacteria-derived extracellular vesicles present in stool at a family level, the content of extracellular vesicles derived from bacteria belonging to the family Spirochaetaceae and the family S24-7 was significantly different between colon cancer patients and colon polyp patients (see Example 6).

More particularly, in one embodiment of the present invention, as a result of performing metagenomic analysis on bacteria-derived extracellular vesicles present in stool at a genus level, the content of extracellular vesicles derived from bacteria belonging to the genus Treponema, the genus Dialister, and the genus Oscillospira was significantly different between colon cancer patients and colon polyp patients (see Example 6).

More particularly, in one embodiment of the present invention, as a result of performing metagenomic analysis on bacteria-derived extracellular vesicles present in urine at an order level, the content of extracellular vesicles derived from bacteria belonging to the order Myxococcales was significantly different between colon cancer patients and colon polyp patients (see Example 7).

More particularly, in one embodiment of the present invention, as a result of performing metagenomic analysis on bacteria-derived extracellular vesicles present in urine at a genus level, the content of extracellular vesicles derived from bacteria belonging to the genus Eubacterium was significantly different between colon cancer patients and colon polyp patients (see Example 7).

More particularly, in one embodiment of the present invention, as a result of performing metagenomic analysis on bacteria-derived extracellular vesicles present in stool at a phylum level, the content of extracellular vesicles derived from bacteria belonging to the phylum Actinobacteria was significantly different between colon polyp patients and normal individuals (see Example 8).

More particularly, in one embodiment of the present invention, as a result of performing metagenomic analysis on bacteria-derived extracellular vesicles present in stool at a class level, the content of extracellular vesicles derived from bacteria belonging to the class Betaproteobacteria and the class Solibacteres was significantly different between colon polyp patients and normal individuals (see Example 8).

More particularly, in one embodiment of the present invention, as a result of performing metagenomic analysis on bacteria-derived extracellular vesicles present in stool at an order level, the content of extracellular vesicles derived from bacteria belonging to the order Burkholderiales, the order Sphingomonadales, and the order Solibacterales was significantly different between colon polyp patients and normal individuals (see Example 8).

More particularly, in one embodiment of the present invention, as a result of performing metagenomic analysis on bacteria-derived extracellular vesicles present in stool at a family level, the content of extracellular vesicles derived from bacteria belonging to the family Rhizobiaceae and the family Sphingomonadaceae was significantly different between colon polyp patients and normal individuals (see Example 8).

More particularly, in one embodiment of the present invention, as a result of performing metagenomic analysis on bacteria-derived extracellular vesicles present in stool at a genus level, the content of extracellular vesicles derived from bacteria belonging to the genus Sphingomonas, the genus Alkanindiges, and the genus Roseateles was significantly different between colon polyp patients and normal individuals (see Example 8).

More particularly, in one embodiment of the present invention, as a result of performing metagenomic analysis on bacteria-derived extracellular vesicles present in urine at a phylum level, the content of extracellular vesicles derived from bacteria belonging to the phylum Proteobacteria and the phylum Euryarchaeota was significantly different between colon polyp patients and normal individuals (see Example 9).

More particularly, in one embodiment of the present invention, as a result of performing metagenomic analysis on bacteria-derived extracellular vesicles present in urine at a class level, the content of extracellular vesicles derived from bacteria belonging to the class Gammaproteobacteria, the class Clostridia, the class Methanobacteria, and the class 4C0d-2 was significantly different between colon polyp patients and normal individuals (see Example 9).

More particularly, in one embodiment of the present invention, as a result of performing metagenomic analysis on bacteria-derived extracellular vesicles present in urine at an order level, the content of extracellular vesicles derived from bacteria belonging to the order Stramenopiles, the order Pseudomonadales, the order Clostridiales, the order Oceanospirillales, the order Desulfovibrionales, and the order Methanobacteriales was significantly different between colon polyp patients and normal individuals (see Example 9).

More particularly, in one embodiment of the present invention, as a result of performing metagenomic analysis on bacteria-derived extracellular vesicles present in urine at a family level, the content of extracellular vesicles derived from bacteria belonging to the family Exiguobacteraceae, the family Moraxellaceae, the family Pseudomonadaceae, the family Rhizobiaceae, the family Streptococcaceae, the family Peptostreptococcaceae, the family Comamonadaceae, the family Veillonellaceae, the family Bacteroidaceae, the family Paraprevotellaceae, the family Ruminococcaceae, the family Corynebacteriaceae, the family Christensenellaceae, the family Odoribacteraceae, the family Desulfovibrionaceae, the family Halomonadaceae, the family Alcaligenaceae, the family Barnesiellaceae, the family Methanobacteriaceae, and the family Rikenellaceae was significantly different between colon polyp patients and normal individuals (see Example 9).

More particularly, in one embodiment of the present invention, as a result of performing metagenomic analysis on bacteria-derived extracellular vesicles present in urine at a genus level, the content of extracellular vesicles derived from bacteria belonging to the genus Rhizobium, the genus Morganella, the genus Proteus, the genus Exiguobacterium, the genus Acinetobacter, the genus Pseudomonas, the genus SMB53, the genus Lactococcus, the genus Coprococcus, the genus Streptococcus, the genus Bacteroides, the genus Ruminococcus, the genus Corynebacterium, the genus Odoribacter, the genus Clostridium, the genus Comamonas, the genus Paraprevotella, the genus Roseburia, the genus Citrobacter, the genus Klebsiella, the genus Virgibacillus, the genus Slackia, the genus Dialister, the genus Phascolarctobacterium, the genus Sutterella, the genus Halomonas, the genus Roseomonas, and the genus Methanobrevibacter was significantly different between colon polyp patients and normal individuals (see Example 9).

From the above-described example results, it was confirmed that bacteria-derived extracellular vesicles exhibiting a significant change in content in colon cancer patients compared to normal individuals and colon polyp patients were identified by performing metagenomic analysis on bacteria-derived extracellular vesicles isolated from stool and urine, and colon cancer could be diagnosed by analyzing an increase or decrease in the content of bacteria-derived extracellular vesicles at each level through metagenomic analysis.

From the above-described example results, it was also confirmed that bacteria-derived extracellular vesicles exhibiting a significant change in content in colon polyp patients compared to normal individuals were identified by performing metagenomic analysis on bacteria-derived extracellular vesicles isolated from stool and urine, and a colon polyp could be diagnosed by analyzing an increase or decrease in the content of bacteria-derived extracellular vesicles at each level through metagenomic analysis.

Hereinafter, the present invention will be described with reference to exemplary examples to aid in understanding of the present invention. However, these examples are provided only for illustrative purposes and are not intended to limit the scope of the present invention.

### MODE OF THE INVENTION

### Examples

### Example 1. Analysis of In Vivo Absorption. Distribution, and Excretion Patterns of Intestinal Bacteria and Bacteria-Derived Extracellular Vesicles

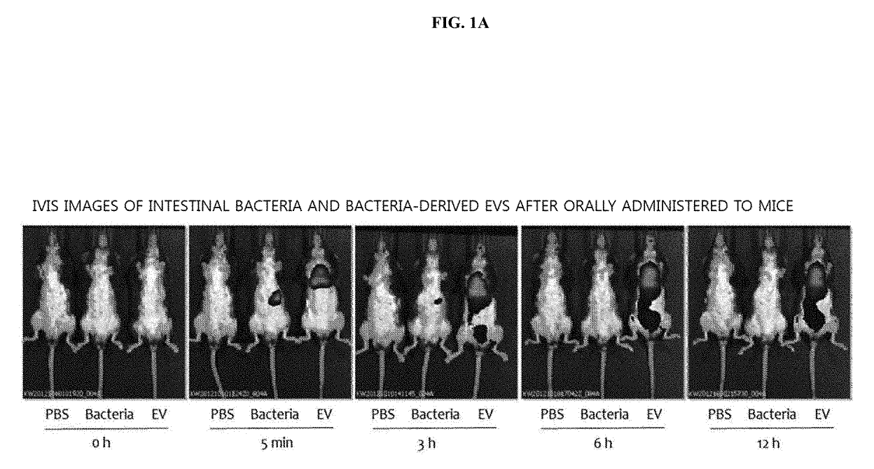

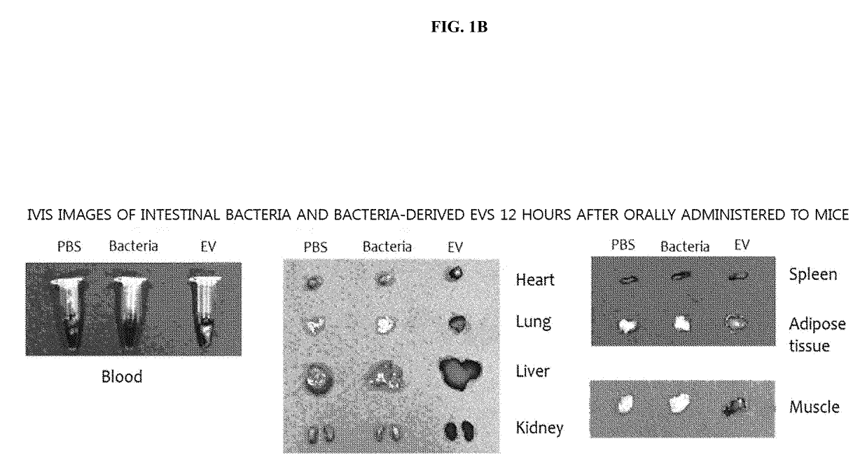

To evaluate whether intestinal bacteria and bacteria-derived extracellular vesicles are systematically absorbed through the gastrointestinal tract, an experiment was conducted using the following method. More particularly, 50 μg of each of intestinal bacteria and the bacteria-derived extracellular vesicles (EVs), labeled with fluorescence, were orally administered to the gastrointestinal tracts of mice, and fluorescence was measured at 0 h, and after 5 min, 3 h, 6 h, and 12 h. As a result of observing the entire images of mice, as illustrated in FIG. 1A, the bacteria were not systematically absorbed when administered, while the bacteria-derived EVs were systematically absorbed at 5 min after administration, and, at 3 h after administration, fluorescence was strongly observed in the bladder, from which it was confirmed that the EVs were excreted via the urinary system, and were present in the bodies up to 12 h after administration.

After intestinal bacteria and intestinal bacteria-derived extracellular vesicles were systematically absorbed, to evaluate a pattern of invasion of intestinal bacteria and the bacteria-derived EVs into various organs in the human body after being systematically absorbed, 50 μg of each of the bacteria and bacteria-derived EVs, labeled with fluorescence, were administered using the same method as that used above, and then, at 12 h after administration, blood, the heart, the lungs, the liver, the kidneys, the spleen, adipose tissue, and muscle were extracted from each mouse. As a result of observing fluorescence in the extracted tissues, as illustrated in FIG. 1B, it was confirmed that the intestinal bacteria were not absorbed into each organ, while the bacteria-derived EVs were distributed in the blood, heart, lungs, liver, kidneys, spleen, adipose tissue, and muscle.

### Example 2. Vesicle Isolation and DNA Extraction from Stool and Urine

To isolate extracellular vesicles and extract DNA, from stool and urine, first, stool and urine was added to a 10 ml tube and centrifuged at 3,500×g and 4° C. for 10 min to precipitate a suspension, and only a supernatant was collected, which was then placed in a new 10 ml tube. The collected supernatant was filtered using a 0.22 μm filter to remove bacteria and impurities, and then placed in centripreigugal filters (50 kD) and centrifuged at 1500×g and 4° C. for 15 min to discard materials with a smaller size than 50 kD, and then concentrated to 10 ml. Once again, bacteria and impurities were removed therefrom using a 0.22 μm filter, and then the resulting concentrate was subjected to ultra-high speed centrifugation at 150,000×g and 4° C. for 3 hours by using a Type 90ti rotor to remove a supernatant, and the agglomerated pellet was dissolved with phosphate-buffered saline (PBS), thereby obtaining vesicles.

100 μl of the extracellular vesicles isolated from the stool and urine according to the above-described method was boiled at 100° C. to allow the internal DNA to come out of the lipid and then cooled on ice. Next, the resulting vesicles were centrifuged at 10,000×g and 4° C. for 30 minutes to remove the remaining suspension, only the supernatant was collected, and then the amount of DNA extracted was quantified using a NanoDrop sprectrophotometer. In addition, to verify whether bacteria-derived DNA was present in the extracted DNA, PCR was performed using 16s rDNA primers shown in Table 1 below.

TABLE 1

| Primer | | Sequence | SEQ ID NO. |
|---|---|---|---|
| 16S rDNA | 16S_V3_F | 5′-TCGTCGGCAGCGTCAGAT | 1 |
| | | GTGTATAAGAGACAGCCTACG | |
| | | GGNGGCWGCAG-3′ | |
| | 16S_V4_R | 5′-GTCTCGTGGGCTCGGAGA | 2 |
| | | TGTGTATAAGAGACAGGACTA | |
| | | CHVGGGTATCTAATCC-3′ | |

### Example 3. Metagenomic Analysis Using DNA Extracted from Vesicle in Stool and Urine

DNA was extracted using the same method as that used in Example 2, and then PCR was performed thereon using 16S rDNA primers shown in Table 1 to amplify DNA, followed by sequencing (Illumina MiSeq sequencer). The results were output as standard flowgram format (SFF) files, and the SFF files were converted into sequence files (.fasta) and nucleotide quality score files using GS FLX software (v2.9), and then credit rating for reads was identified, and portions with a window (20 bps) average base call accuracy of less than 99% (Phred score <20) were removed. After removing the low-quality portions, only reads having a length of 300 bps or more were used (Sickle version 1.33), and, for operational taxonomy unit (OTU) analysis, clustering was performed using UCLUST and USEARCH according to sequence similarity. In particular, clustering was performed based on sequence similarity values of 94% for genus, 90% for family, 85% for order, 80% for class, and 75% for phylum, and phylum, class, order, family, and genus levels of each OTU were classified, and bacteria with a sequence similarity of 97% or more were analyzed (QIIME) using 16S DNA sequence databases (108,453 sequences) of BLASTN and GreenGenes.

### Example 4. Colon Cancer Diagnostic Model Based on Metagenomic Analysis of Bacteria-Derived EVs Isolated from Stool of Normal Individuals and Colon Cancer Patients

EVs were isolated from stool samples of 29 colon cancer patients and 358 normal individuals, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

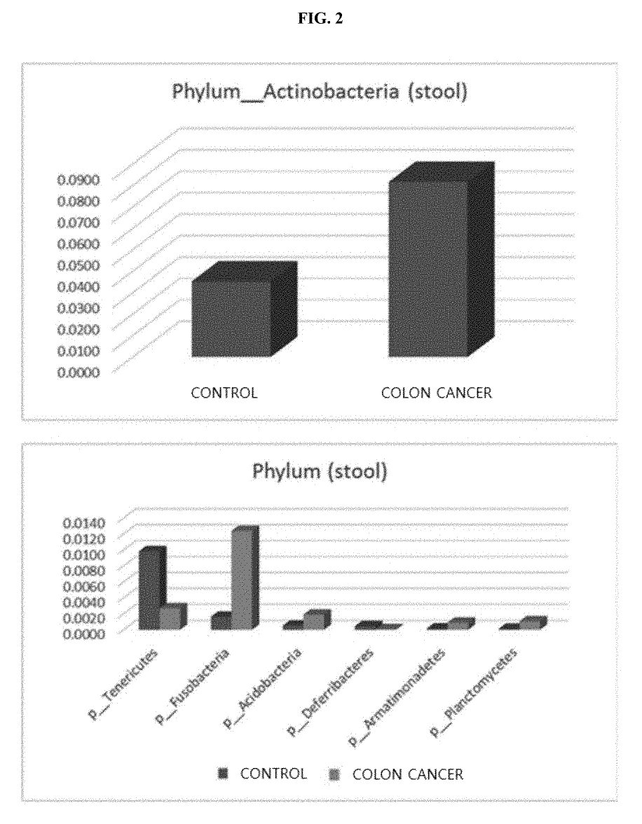

As a result of analyzing bacteria-derived extracellular vesicles in stool at a phylum level, a diagnostic model developed using, as a biomarker, one or more bacteria from the phylum Deferribacteres, the phylum Tenericutes, the phylum Actinobacteria, the phylum Acidobacteria, the phylum Armatimonadetes, the phylum Planctomycetes, and the phylum Fusobacteria exhibited significant diagnostic performance for colon cancer (see Table 2 and FIG. 2).

TABLE 2

| | Control | | Colon Cancer | | t-test | | | | |
|---|---|---|---|---|---|---|---|---|---|
| Taxon | Mean | SD | Mean | SD | p-value | Ratio | AUC | sensitivity | specificity |
| p_Deferribacteres | 0.0004 | 0.0018 | 0.0001 | 0.0003 | 0.0006 | 0.16 | 0.74 | 1.00 | 0.03 |
| p_Tenericutes | 0.0098 | 0.0256 | 0.0027 | 0.0064 | 0.0001 | 0.27 | 0.74 | 1.00 | 0.03 |
| p_Actinobacteria | 0.0353 | 0.0403 | 0.0817 | 0.0588 | 0.0003 | 2.32 | 0.80 | 0.99 | 0.14 |
| p_Acidobacteria | 0.0005 | 0.0024 | 0.0019 | 0.0021 | 0.0024 | 3.61 | 0.75 | 1.00 | 0.03 |
| p_Armatimonadetes | 0.0002 | 0.0007 | 0.0009 | 0.0013 | 0.0075 | 5.11 | 0.77 | 1.00 | 0.07 |
| p_Planctomycetes | 0.0001 | 0.0006 | 0.0011 | 0.0015 | 0.0027 | 7.36 | 0.82 | 0.99 | 0.24 |
| p_Fusobacteria | 0.0016 | 0.0040 | 0.0124 | 0.0202 | 0.0088 | 7.60 | 0.82 | 0.99 | 0.17 |

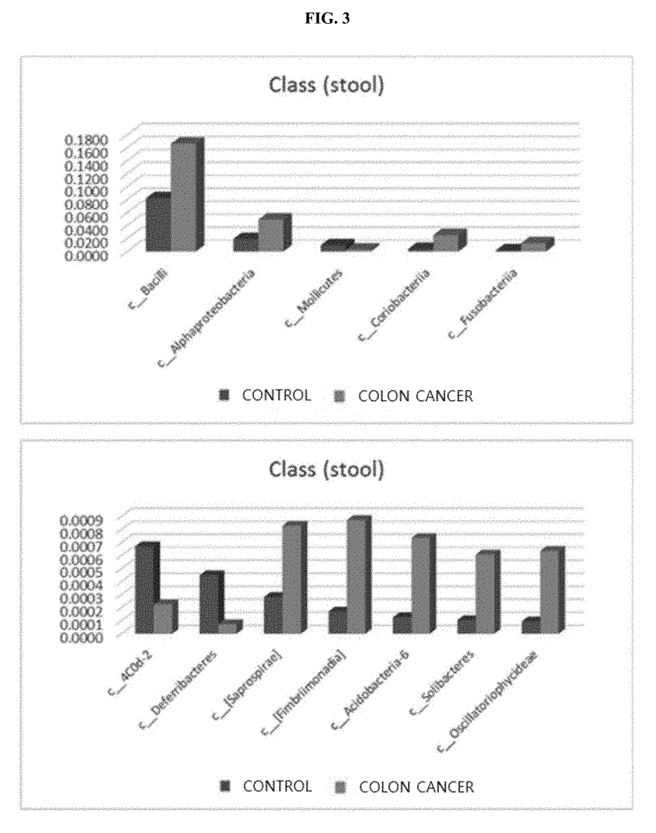

As a result of analyzing bacteria-derived extracellular vesicles in stool at a class level, a diagnostic model developed using, as a biomarker, one or more bacteria from the class Deferribacteres, the class Mollicutes, the class 4C0d-2, the class Bacilli, the class Alphaproteobacteria, the class Saprospirae, the class Fimbriimonadia, the class Acidobacteria-6, the class Solibacteres, the class Coriobacteriia, the class Oscillatoriophycideae, and the class Fusobacteriia exhibited significant diagnostic performance for colon cancer (see Table 3 and FIG. 3).

TABLE 3

| | Control | | Colon Cancer | | t-test | | | | |
|---|---|---|---|---|---|---|---|---|---|
| Taxon | Mean | SD | Mean | SD | p-value | Ratio | AUC | sensitivity | specificity |
| c_Deferribacteres | 0.0004 | 0.0018 | 0.0001 | 0.0003 | 0.0006 | 0.16 | 0.74 | 1.00 | 0.03 |
| c_Mollicutes | 0.0095 | 0.0254 | 0.0026 | 0.0064 | 0.0002 | 0.28 | 0.74 | 1.00 | 0.03 |
| c_4C0d-2 | 0.0007 | 0.0028 | 0.0002 | 0.0004 | 0.0084 | 0.34 | 0.73 | 1.00 | 0.03 |
| c_Bacilli | 0.0820 | 0.0955 | 0.1661 | 0.1537 | 0.0078 | 2.03 | 0.75 | 1.00 | 0.03 |
| c_Alphaproteobacteria | 0.0192 | 0.0306 | 0.0491 | 0.0520 | 0.0054 | 2.56 | 0.80 | 1.00 | 0.14 |
| c_[Saprospirae] | 0.0003 | 0.0010 | 0.0008 | 0.0012 | 0.0040 | 2.94 | 0.74 | 1.00 | 0.03 |
| c_[Fimbriimonadia] | 0.0002 | 0.0007 | 0.0009 | 0.0013 | 0.0075 | 5.14 | 0.77 | 1.00 | 0.07 |
| c_Acidobacteria-6 | 0.0001 | 0.0007 | 0.0007 | 0.0010 | 0.0037 | 5.85 | 0.78 | 0.99 | 0.03 |
| c_Solibacteres | 0.0001 | 0.0006 | 0.0006 | 0.0009 | 0.0090 | 5.86 | 0.75 | 0.99 | 0.07 |
| c_Coriobacteriia | 0.0040 | 0.0081 | 0.0251 | 0.0363 | 0.0046 | 6.33 | 0.80 | 0.99 | 0.17 |
| c_Oscillatoriophycideae | 0.0001 | 0.0005 | 0.0006 | 0.0009 | 0.0050 | 6.72 | 0.80 | 0.99 | 0.10 |
| c_Fusobacteriia | 0.0016 | 0.0040 | 0.0124 | 0.0202 | 0.0088 | 7.60 | 0.82 | 0.99 | 0.17 |

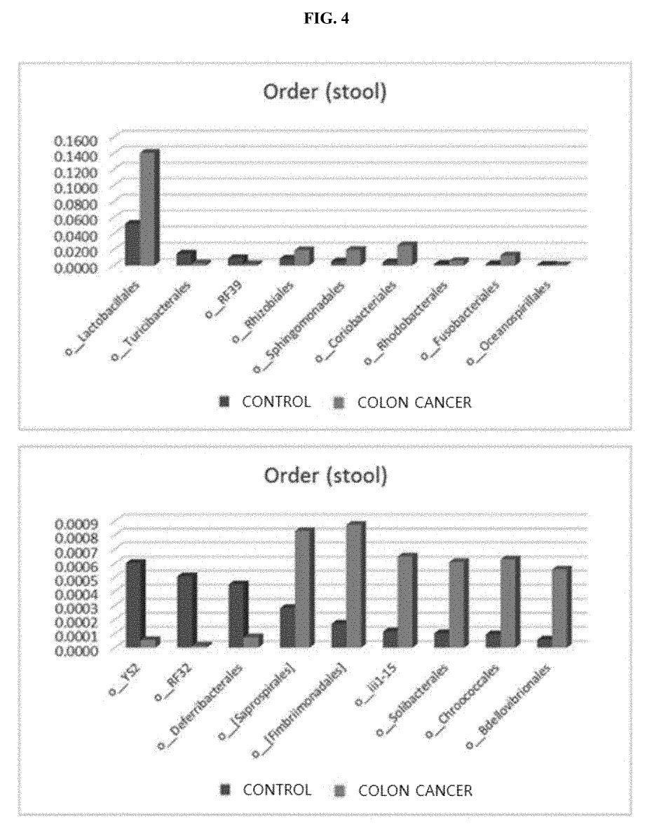

As a result of analyzing bacteria-derived extracellular vesicles in stool at an order level, a diagnostic model developed using, as a biomarker, one or more bacteria from the order RF32, the order YS2, the order Deferribacterales, the order Turicibacterales, the order RF39, the order Oceanospirillales, the order Rhizobiales, the order Lactobacillales, the order Rhodobacterales, the order Saprospirales, the order Sphingomonadales, the order Fimbriimonadales, the order iii1-15, the order Solibacterales, the order Coriobacteriales, the order Chroococcales, the order Fusobacteriales, and the order Bdellovibrionales exhibited significant diagnostic performance for colon cancer (see Table 4 and FIG. 4).

TABLE 4

| | Control | | Colon Cancer | | t-test | | | | |
|---|---|---|---|---|---|---|---|---|---|
| Taxon | Mean | SD | Mean | SD | p-value | Ratio | AUC | sensitivity | specificity |
| o_RF32 | 0.0005 | 0.0026 | 0.0000 | 0.0001 | 0.0005 | 0.03 | 0.75 | 1.00 | 0.03 |
| o_YS2 | 0.0006 | 0.0027 | 0.0001 | 0.0002 | 0.0003 | 0.09 | 0.74 | 1.00 | 0.03 |
| o_Deferribacterales | 0.0004 | 0.0018 | 0.0001 | 0.0003 | 0.0006 | 0.16 | 0.74 | 1.00 | 0.03 |
| o_Turicibacterales | 0.0148 | 0.0493 | 0.0024 | 0.0053 | 0.0000 | 0.16 | 0.75 | 1.00 | 0.03 |
| o_RF39 | 0.0090 | 0.0248 | 0.0017 | 0.0046 | 0.0000 | 0.18 | 0.75 | 1.00 | 0.03 |
| o_Oceanospirillales | 0.0009 | 0.0033 | 0.0002 | 0.0003 | 0.0001 | 0.22 | 0.75 | 1.00 | 0.03 |
| o_Rhizobiales | 0.0085 | 0.0202 | 0.0189 | 0.0215 | 0.0082 | 2.23 | 0.75 | 1.00 | 0.03 |
| o_Lactobacillales | 0.0519 | 0.0809 | 0.1402 | 0.1495 | 0.0044 | 2.70 | 0.76 | 1.00 | 0.10 |
| o_Rhodobacterales | 0.0019 | 0.0057 | 0.0054 | 0.0059 | 0.0020 | 2.81 | 0.76 | 1.00 | 0.03 |
| o_[Saprospirales] | 0.0003 | 0.0010 | 0.0008 | 0.0012 | 0.0040 | 2.94 | 0.74 | 1.00 | 0.03 |
| o_Sphingomonadales | 0.0050 | 0.0097 | 0.0192 | 0.0207 | 0.0012 | 3.87 | 0.85 | 0.99 | 0.21 |
| o_[Fimbriimonadales] | 0.0002 | 0.0007 | 0.0009 | 0.0013 | 0.0075 | 5.14 | 0.77 | 1.00 | 0.07 |
| o_iii1-15 | 0.0001 | 0.0007 | 0.0006 | 0.0010 | 0.0090 | 5.67 | 0.78 | 0.99 | 0.03 |
| o_Solibacterales | 0.0001 | 0.0006 | 0.0006 | 0.0009 | 0.0089 | 5.89 | 0.75 | 0.99 | 0.07 |
| o_Coriobacteriales | 0.0040 | 0.0081 | 0.0251 | 0.0363 | 0.0046 | 6.33 | 0.80 | 0.99 | 0.17 |
| o_Chroococcales | 0.0001 | 0.0005 | 0.0006 | 0.0009 | 0.0050 | 6.63 | 0.80 | 0.99 | 0.14 |
| o_Fusobacteriales | 0.0016 | 0.0040 | 0.0124 | 0.0202 | 0.0088 | 7.60 | 0.82 | 0.99 | 0.17 |
| o_Bdellovibrionales | 0.0001 | 0.0004 | 0.0006 | 0.0009 | 0.0063 | 9.79 | 0.76 | 0.99 | 0.10 |

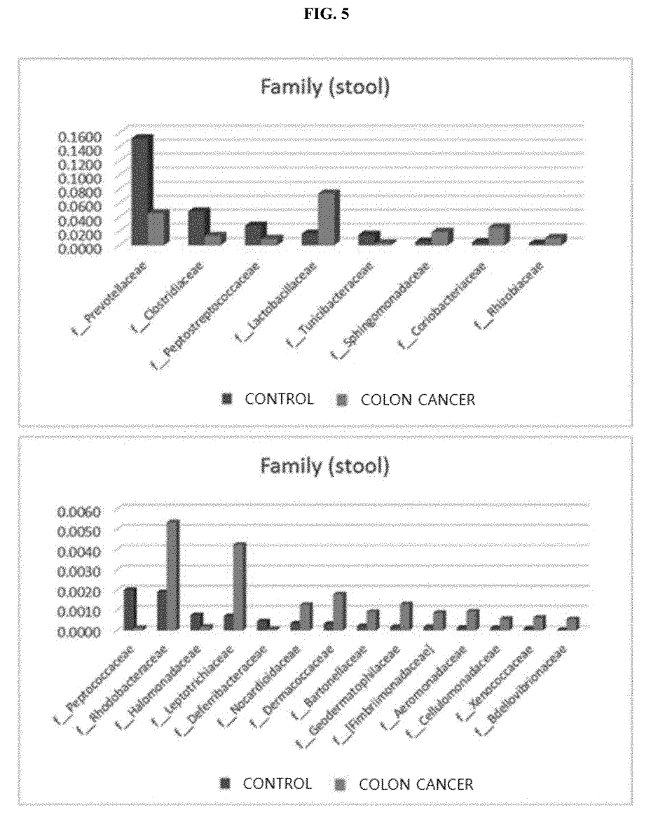

As a result of analyzing bacteria-derived extracellular vesicles in stool at a family level, a diagnostic model developed using, as a biomarker, one or more bacteria from the family Peptococcaceae, the family Deferribacteraceae, the family Turicibacteraceae, the family Halomonadaceae, the family Clostridiaceae, the family Prevotellaceae, the family Peptostreptococcaceae, the family Rhodobacteraceae, the family Nocardioidaceae, the family Sphingomonadaceae, the family Bartonellaceae, the family Cellulomonadaceae, the family Lactobacillaceae, the family Rhizobiaceae, the family Fimbriimonadaceae, the family Dermacoccaceae, the family Leptotrichiaceae, the family Coriobacteriaceae, the family Xenococcaceae, the family Aeromonadaceae, the family Geodermatophilaceae, and the family Bdellovibrionaceae exhibited significant diagnostic performance for colon cancer (see Table 5 and FIG. 5).

TABLE 5

| | Control | | Colon Cancer | | t-test | | | | |
|---|---|---|---|---|---|---|---|---|---|
| Taxon | Mean | SD | Mean | SD | p-value | Ratio | AUC | sensitivity | specificity |
| f_Peptococcaceae | 0.0020 | 0.0048 | 0.0001 | 0.0003 | 0.0000 | 0.07 | 0.80 | 1.00 | 0.03 |
| f_Deferribacteraceae | 0.0004 | 0.0018 | 0.0001 | 0.0003 | 0.0006 | 0.16 | 0.74 | 1.00 | 0.03 |
| f_Turicibacteraceae | 0.0148 | 0.0493 | 0.0024 | 0.0053 | 0.0000 | 0.16 | 0.75 | 1.00 | 0.03 |
| f_Halomonadaceae | 0.0008 | 0.0027 | 0.0002 | 0.0003 | 0.0002 | 0.25 | 0.74 | 1.00 | 0.03 |
| f_Clostridiaceae | 0.0477 | 0.0792 | 0.0129 | 0.0176 | 0.0000 | 0.27 | 0.79 | 1.00 | 0.03 |
| f_Prevotellaceae | 0.1518 | 0.1717 | 0.0454 | 0.0656 | 0.0000 | 0.30 | 0.79 | 1.00 | 0.03 |
| f_Peptostreptococcaceae | 0.0275 | 0.0623 | 0.0088 | 0.0134 | 0.0000 | 0.32 | 0.76 | 1.00 | 0.03 |
| f_Rhodobacteraceae | 0.0019 | 0.0057 | 0.0054 | 0.0059 | 0.0020 | 2.82 | 0.76 | 1.00 | 0.03 |
| f_Nocardioidaceae | 0.0004 | 0.0016 | 0.0013 | 0.0019 | 0.0037 | 3.58 | 0.75 | 1.00 | 0.03 |
| f_Sphingomonadaceae | 0.0047 | 0.0091 | 0.0187 | 0.0202 | 0.0010 | 4.00 | 0.86 | 0.99 | 0.21 |
| f_Bartonellaceae | 0.0002 | 0.0014 | 0.0009 | 0.0012 | 0.0088 | 4.27 | 0.75 | 1.00 | 0.03 |
| f_Cellulomonadaceae | 0.0001 | 0.0009 | 0.0006 | 0.0009 | 0.0084 | 4.48 | 0.74 | 1.00 | 0.03 |
| f_Lactobacillaceae | 0.0162 | 0.0224 | 0.0733 | 0.0967 | 0.0042 | 4.52 | 0.83 | 0.99 | 0.21 |
| f_Rhizobiaceae | 0.0022 | 0.0040 | 0.0102 | 0.0125 | 0.0023 | 4.62 | 0.83 | 1.00 | 0.28 |
| f_[Fimbriimonadaceae] | 0.0002 | 0.0007 | 0.0009 | 0.0013 | 0.0075 | 5.14 | 0.77 | 1.00 | 0.07 |
| f_Dermacoccaceae | 0.0003 | 0.0010 | 0.0018 | 0.0025 | 0.0047 | 5.58 | 0.81 | 0.99 | 0.14 |
| f_Leptotrichiaceae | 0.0007 | 0.0027 | 0.0042 | 0.0055 | 0.0021 | 5.93 | 0.82 | 0.99 | 0.07 |
| f_Coriobacteriaceae | 0.0040 | 0.0081 | 0.0251 | 0.0363 | 0.0046 | 6.33 | 0.80 | 0.99 | 0.17 |
| f_Xenococcaceae | 0.0001 | 0.0005 | 0.0006 | 0.0009 | 0.0049 | 6.72 | 0.81 | 0.99 | 0.14 |
| f_Aeromonadaceae | 0.0001 | 0.0006 | 0.0009 | 0.0013 | 0.0035 | 7.11 | 0.82 | 0.99 | 0.10 |
| f_Geodermatophilaceae | 0.0002 | 0.0008 | 0.0013 | 0.0018 | 0.0023 | 7.46 | 0.83 | 0.99 | 0.17 |
| f_Bdellovibrionaceae | 0.0000 | 0.0002 | 0.0006 | 0.0009 | 0.0038 | 23.48 | 0.80 | 1.00 | 0.24 |

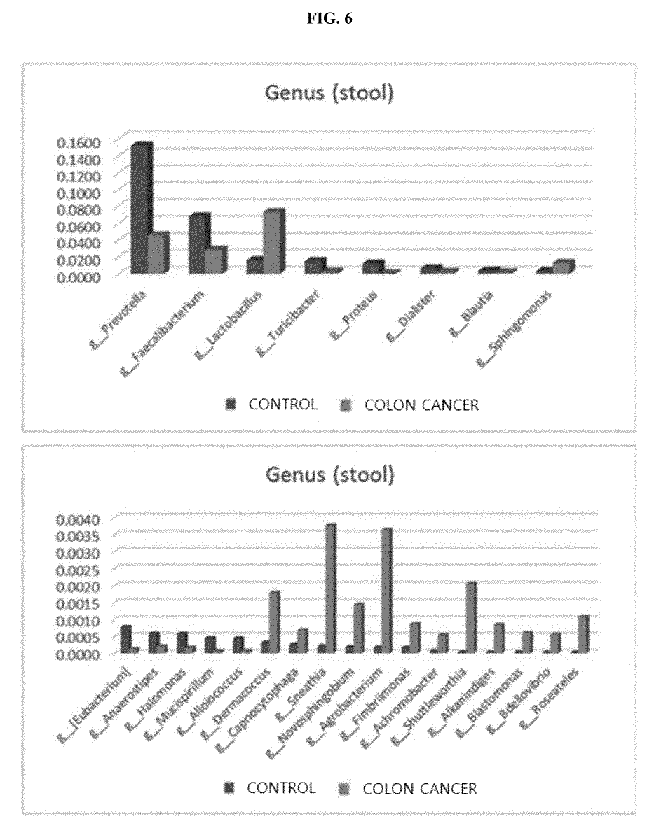

As a result of analyzing bacteria-derived extracellular vesicles in stool at a genus level, a diagnostic model developed using, as a biomarker, one or more bacteria from the genus rc4-4, the genus Proteus, the genus Catenibacterium, the genus Mucispirillum, the genus Eubacterium, the genus Turicibacter, the genus Alloiococcus, the genus Halomonas, the genus Prevotella, the genus Dialister, the genus Anaerostipes, the genus SMB53, the genus Faecalibacterium, the genus Blautia, the genus Capnocytophaga, the genus Sphingomonas, the genus Lactobacillus, the genus Fimbriimonas, the genus Dermacoccus, the genus Achromobacter, the genus Novosphingobium, the genus Sneathia, the genus Agrobacterium, the genus Blastomonas, the genus Bdellovibrio, the genus Alkanindiges, the genus Roseateles, and the genus Shuttleworthia exhibited significant diagnostic performance for colon cancer (see Table 6 and FIG. 6).

TABLE 6

| | Control | | Colon Cancer | | t-test | | | | |
|---|---|---|---|---|---|---|---|---|---|
| Taxon | Mean | SD | Mean | SD | p-value | Ratio | AUC | sensitivity | specificity |
| g_rc4-4 | 0.0010 | 0.0035 | 0.0000 | 0.0000 | 0.0000 | 0.01 | 0.80 | 1.00 | 0.03 |
| g_Proteus | 0.0121 | 0.0272 | 0.0005 | 0.0011 | 0.0000 | 0.04 | 0.80 | 1.00 | 0.03 |
| g_Catenibacterium | 0.0014 | 0.0047 | 0.0002 | 0.0004 | 0.0000 | 0.11 | 0.75 | 1.00 | 0.03 |
| g_Mucispirillum | 0.0004 | 0.0018 | 0.0001 | 0.0003 | 0.0006 | 0.16 | 0.74 | 1.00 | 0.03 |
| g_[Eubacterium] | 0.0008 | 0.0020 | 0.0001 | 0.0003 | 0.0000 | 0.16 | 0.74 | 1.00 | 0.03 |
| g_Turicibacter | 0.0148 | 0.0493 | 0.0024 | 0.0053 | 0.0000 | 0.16 | 0.75 | 1.00 | 0.03 |
| g_Alloiococcus | 0.0004 | 0.0025 | 0.0001 | 0.0002 | 0.0086 | 0.16 | 0.73 | 1.00 | 0.03 |
| g_Halomonas | 0.0006 | 0.0024 | 0.0002 | 0.0003 | 0.0027 | 0.29 | 0.73 | 1.00 | 0.03 |
| g_Prevotella | 0.1518 | 0.1717 | 0.0454 | 0.0656 | 0.0000 | 0.30 | 0.79 | 1.00 | 0.03 |
| g_Dialister | 0.0063 | 0.0186 | 0.0020 | 0.0027 | 0.0001 | 0.32 | 0.75 | 1.00 | 0.03 |
| g_Anaerostipes | 0.0006 | 0.0014 | 0.0002 | 0.0005 | 0.0020 | 0.36 | 0.74 | 1.00 | 0.03 |
| g_SMB53 | 0.0011 | 0.0018 | 0.0004 | 0.0007 | 0.0002 | 0.39 | 0.76 | 1.00 | 0.00 |
| g_Faecalibacterium | 0.0681 | 0.0882 | 0.0285 | 0.0584 | 0.0020 | 0.42 | 0.77 | 1.00 | 0.03 |
| g_Blautia | 0.0037 | 0.0100 | 0.0017 | 0.0017 | 0.0009 | 0.45 | 0.74 | 1.00 | 0.03 |
| g_Capnocytophaga | 0.0003 | 0.0008 | 0.0007 | 0.0009 | 0.0052 | 2.59 | 0.75 | 1.00 | 0.03 |
| g_Sphingomonas | 0.0032 | 0.0059 | 0.0128 | 0.0138 | 0.0011 | 3.95 | 0.85 | 0.99 | 0.28 |
| g_Lactobacillus | 0.0160 | 0.0223 | 0.0731 | 0.0966 | 0.0041 | 4.58 | 0.83 | 0.99 | 0.21 |
| g_Fimbriimonas | 0.0002 | 0.0007 | 0.0009 | 0.0013 | 0.0079 | 5.10 | 0.77 | 1.00 | 0.07 |
| g_Dermacoccus | 0.0003 | 0.0010 | 0.0018 | 0.0025 | 0.0047 | 5.58 | 0.81 | 0.99 | 0.14 |
| g_Achromobacter | 0.0001 | 0.0003 | 0.0005 | 0.0008 | 0.0065 | 6.93 | 0.83 | 0.99 | 0.17 |
| g_Novosphingobium | 0.0002 | 0.0009 | 0.0014 | 0.0021 | 0.0046 | 7.84 | 0.82 | 0.99 | 0.10 |
| g_Sneathia | 0.0002 | 0.0019 | 0.0038 | 0.0050 | 0.0009 | 17.60 | 0.85 | 1.00 | 0.28 |
| g_Agrobacterium | 0.0002 | 0.0006 | 0.0036 | 0.0047 | 0.0005 | 20.68 | 0.87 | 1.00 | 0.41 |
| g_Blastomonas | 0.0000 | 0.0002 | 0.0006 | 0.0008 | 0.0012 | 22.87 | 0.84 | 0.99 | 0.31 |
| g_Bdellovibrio | 0.0000 | 0.0002 | 0.0006 | 0.0009 | 0.0038 | 23.63 | 0.80 | 1.00 | 0.24 |
| g_Alkanindiges | 0.0000 | 0.0002 | 0.0008 | 0.0014 | 0.0037 | 26.94 | 0.85 | 1.00 | 0.28 |
| g_Roseateles | 0.0000 | 0.0002 | 0.0011 | 0.0020 | 0.0086 | 51.47 | 0.83 | 0.99 | 0.31 |
| g_Shuttleworthia | 0.0000 | 0.0003 | 0.0020 | 0.0034 | 0.0043 | 51.55 | 0.84 | 1.00 | 0.34 |

### Example 5. Colon Cancer Diagnostic Model Based on Metagenomic Analysis of Bacteria-Derived EVs Isolated from Urine of Normal Individuals and Colon Cancer Patients

Extracellular vesicles were isolated from urine samples of 38 colon cancer patients and 38 normal individuals, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

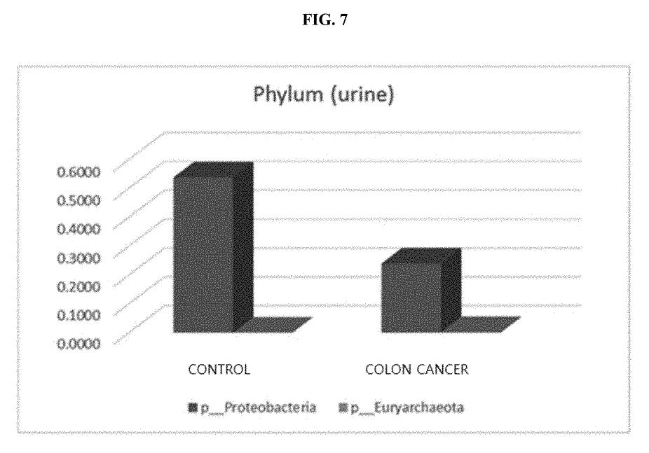

As a result of analyzing bacteria-derived extracellular vesicles in urine at a phylum level, a diagnostic model developed using, as a biomarker, one or more bacteria from the phylum Proteobacteria and the phylum Euryarchaeota exhibited significant diagnostic performance for colon cancer (see Table 7 and FIG. 7).

TABLE 7

| | Control | | Colon Cancer | | t-test | | | | |
|---|---|---|---|---|---|---|---|---|---|
| Taxon | Mean | SD | Mean | SD | p-value | Ratio | AUC | sensitivity | specificity |
| p_Proteobacteria | 0.5379 | 0.1871 | 0.2394 | 0.0820 | 0.0000 | 0.45 | 0.94 | 0.84 | 0.87 |
| p_Euryarchaeota | 0.0000 | 0.0001 | 0.0021 | 0.0021 | 0.0000 | 70.49 | 0.90 | 0.97 | 0.71 |

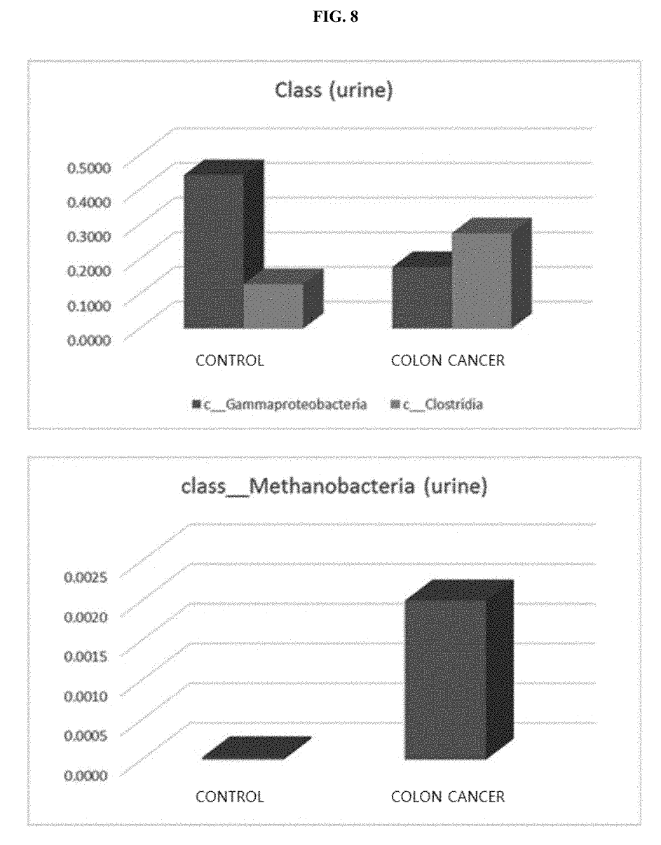

As a result of analyzing bacteria-derived extracellular vesicles in urine at a class level, a diagnostic model developed using, as a biomarker, one or more bacteria from the class Gammaproteobacteria, the class Clostridia, and the class Methanobacteria exhibited significant diagnostic performance for colon cancer (see Table 8 and FIG. 8).

TABLE 8

| | Control | | Colon Cancer | | t-test | | | | |
|---|---|---|---|---|---|---|---|---|---|
| Taxon | Mean | SD | Mean | SD | p-value | Ratio | AUC | sensitivity | specificity |
| c_Gammaproteobacteria | 0.4431 | 0.2104 | 0.1776 | 0.0577 | 0.0000 | 0.40 | 0.90 | 0.76 | 0.87 |
| c_Clostridia | 0.1286 | 0.0723 | 0.2752 | 0.0844 | 0.0000 | 2.14 | 0.93 | 0.87 | 0.82 |
| c_Methanobacteria | 0.0000 | 0.0001 | 0.0020 | 0.0021 | 0.0000 | 67.36 | 0.90 | 0.97 | 0.68 |

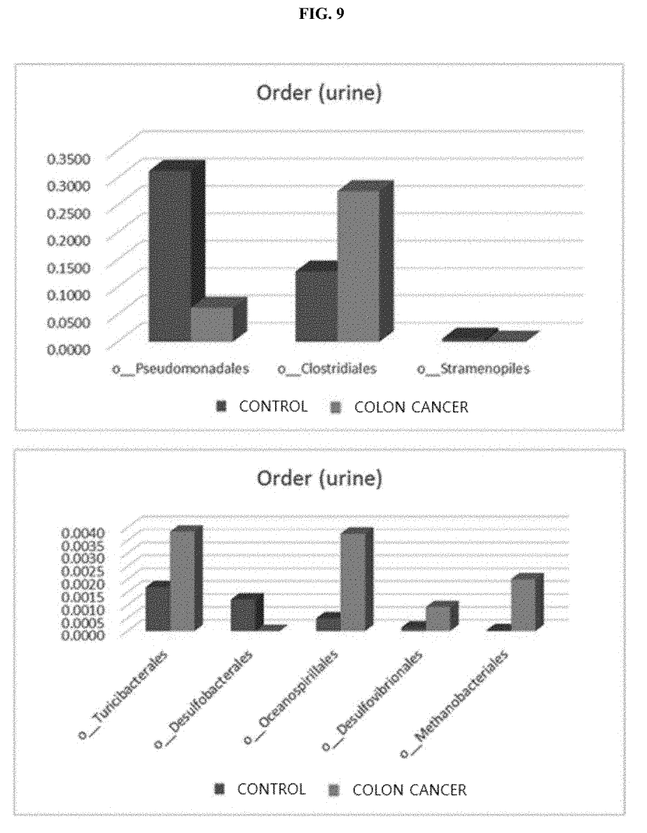

As a result of analyzing bacteria-derived extracellular vesicles in urine at an order level, a diagnostic model developed using, as a biomarker, one or more bacteria from the class Desulfobacterales, the class Stramenopiles, the class Pseudomonadales, the class Clostridiales, the class Turicibacterales, the class Desulfovibrionales, the class Oceanospirillales, and the class Methanobacteriales exhibited significant diagnostic performance for colon cancer (see Table 9 and FIG. 9).

TABLE 9

| | Control | | Colon Cancer | | t-test | | | | |
|---|---|---|---|---|---|---|---|---|---|
| Taxon | Mean | SD | Mean | SD | p-value | Ratio | AUC | sensitivity | specificity |
| o_Desulfobacterales | 0.0012 | 0.0027 | 0.0000 | 0.0000 | 0.0078 | 0.00 | 0.77 | 0.53 | 0.76 |
| o_Stramenopiles | 0.0055 | 0.0083 | 0.0001 | 0.0004 | 0.0003 | 0.02 | 0.82 | 0.58 | 0.87 |
| o_Pseudomonadales | 0.3124 | 0.1910 | 0.0622 | 0.0401 | 0.0000 | 0.20 | 0.93 | 0.87 | 0.92 |
| o_Clostridiales | 0.1284 | 0.0725 | 0.2751 | 0.0843 | 0.0000 | 2.14 | 0.93 | 0.87 | 0.82 |
| o_Turicibacterales | 0.0017 | 0.0026 | 0.0038 | 0.0040 | 0.0075 | 2.27 | 0.72 | 0.71 | 0.61 |
| o_Desulfovibrionales | 0.0001 | 0.0005 | 0.0009 | 0.0010 | 0.0001 | 6.78 | 0.79 | 0.89 | 0.58 |
| o_Oceanospirillales | 0.0005 | 0.0012 | 0.0038 | 0.0073 | 0.0098 | 7.70 | 0.84 | 0.87 | 0.58 |
| o_Methanobacteriales | 0.0000 | 0.0001 | 0.0020 | 0.0021 | 0.0000 | 67.36 | 0.90 | 0.97 | 0.68 |

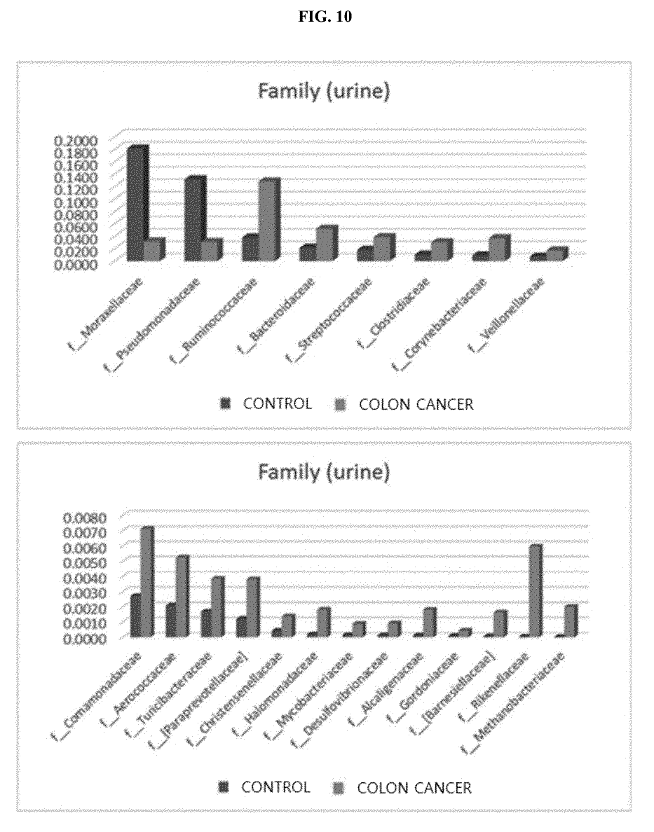

As a result of analyzing bacteria-derived extracellular vesicles in urine at a family level, a diagnostic model developed using, as a biomarker, one or more bacteria from the family Moraxellaceae, the family Pseudomonadaceae, the family Streptococcaceae, the family Turicibacteraceae, the family Veillonellaceae, the family Bacteroidaceae, the family Aerococcaceae, the family Comamonadaceae, the family Clostridiaceae, the family Paraprevotellaceae, the family Christensenellaceae, the family Ruminococcaceae, the family Corynebacteriaceae, the family Gordoniaceae, the family Mycobacteriaceae, the family Desulfovibrionaceae, the family Halomonadaceae, the family Alcaligenaceae, the family Barnesiellaceae, the family Methanobacteriaceae, and the family Rikenellaceaeexhibited significant diagnostic performance for colon cancer (see Table 10 and FIG. 10).

TABLE 10

| | Control | | Colon Cancer | | t-test | | | | |
|---|---|---|---|---|---|---|---|---|---|
| Taxon | Mean | SD | Mean | SD | p-value | Ratio | AUC | sensitivity | specificity |
| f_Moraxellaceae | 0.1810 | 0.1401 | 0.0313 | 0.0220 | 0.0000 | 0.17 | 0.93 | 0.87 | 0.87 |
| f_Pseudomonadaceae | 0.1314 | 0.0973 | 0.0306 | 0.0204 | 0.0000 | 0.23 | 0.90 | 0.82 | 0.89 |
| f_Streptococcaceae | 0.0180 | 0.0126 | 0.0383 | 0.0167 | 0.0000 | 2.13 | 0.89 | 0.79 | 0.87 |
| f_Turicibacteraceae | 0.0017 | 0.0026 | 0.0038 | 0.0040 | 0.0075 | 2.27 | 0.72 | 0.71 | 0.61 |
| f_Veillonellaceae | 0.0070 | 0.0083 | 0.0168 | 0.0074 | 0.0000 | 2.41 | 0.85 | 0.71 | 0.74 |
| f_Bacteroidaceae | 0.0212 | 0.0207 | 0.0517 | 0.0381 | 0.0001 | 2.44 | 0.84 | 0.82 | 0.74 |
| f_Aerococcaceae | 0.0021 | 0.0033 | 0.0052 | 0.0059 | 0.0057 | 2.51 | 0.74 | 0.74 | 0.61 |
| f_Comamonadaceae | 0.0027 | 0.0036 | 0.0071 | 0.0062 | 0.0003 | 2.63 | 0.76 | 0.76 | 0.63 |
| f_Clostridiaceae | 0.0100 | 0.0114 | 0.0299 | 0.0205 | 0.0000 | 2.99 | 0.86 | 0.79 | 0.71 |
| f_[Paraprevotellaceae] | 0.0012 | 0.0036 | 0.0038 | 0.0033 | 0.0017 | 3.11 | 0.82 | 0.82 | 0.63 |
| f_Christensenellaceae | 0.0004 | 0.0013 | 0.0014 | 0.0014 | 0.0040 | 3.16 | 0.74 | 0.87 | 0.68 |
| f_Ruminococcaceae | 0.0379 | 0.0431 | 0.1280 | 0.0390 | 0.0000 | 3.38 | 0.95 | 0.92 | 0.87 |
| f_Corynebacteriaceae | 0.0090 | 0.0097 | 0.0366 | 0.0197 | 0.0000 | 4.07 | 0.96 | 0.89 | 0.87 |
| f_Gordoniaceae | 0.0001 | 0.0005 | 0.0004 | 0.0006 | 0.0081 | 5.03 | 0.78 | 0.76 | 0.68 |
| f_Mycobacteriaceae | 0.0002 | 0.0008 | 0.0009 | 0.0015 | 0.0090 | 5.82 | 0.76 | 0.92 | 0.58 |
| f_Desulfovibrionaceae | 0.0001 | 0.0005 | 0.0009 | 0.0010 | 0.0001 | 6.78 | 0.79 | 0.89 | 0.58 |
| f_Halomonadaceae | 0.0002 | 0.0008 | 0.0018 | 0.0022 | 0.0001 | 9.40 | 0.88 | 0.92 | 0.66 |
| f_Alcaligenaceae | 0.0001 | 0.0004 | 0.0018 | 0.0017 | 0.0000 | 15.77 | 0.90 | 0.95 | 0.79 |
| f_[Barnesiellaceae] | 0.0001 | 0.0004 | 0.0016 | 0.0018 | 0.0000 | 24.08 | 0.84 | 0.95 | 0.66 |
| f_Methanobacteriaceae | 0.0000 | 0.0001 | 0.0020 | 0.0021 | 0.0000 | 67.36 | 0.90 | 0.97 | 0.68 |
| f_Rikenellaceae | 0.0000 | 0.0002 | 0.0059 | 0.0040 | 0.0000 | 153.18 | 0.98 | 0.97 | 0.92 |

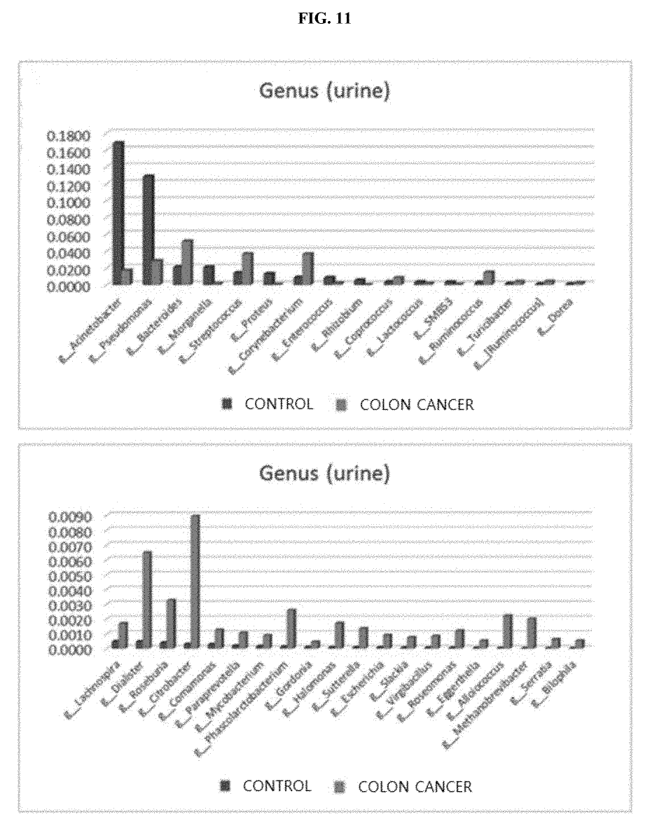

As a result of analyzing bacteria-derived extracellular vesicles in urine at a genus level, a diagnostic model developed using, as a biomarker, one or more bacteria from the genus Rhizobium, the genus Proteus, the genus Morganella, the genus Acinetobacter, the genus Pseudomonas, the genus SMB53, the genus Enterococcus, the genus Lactococcus, the genus Turicibacter, the genus Coprococcus, the genus Bacteroides, the genus Dorea, the genus Streptococcus, the genus Lachnospira, the genus Ruminococcus, the genus Corynebacterium, the genus Comamonas, the genus Gordonia, the genus Paraprevotella, the genus Mycobacterium, the genus Roseburia, the genus Dialister, the genus Slackia, the genus Escherichia, the genus Phascolarctobacterium, the genus Sutterella, the genus Virgibacillus, the genus Eggerthella, the genus Halomonas, the genus Citrobacter, the genus Roseomonas, the genus Alloiococcus, the genus Serratia, the genus Methanobrevibacter, and the genus Bilophila significant diagnostic performance for colon cancer (see Table 11 and FIG. 11).

TABLE 11

| | Control | | Colon Cancer | | t-test | | | | |
|---|---|---|---|---|---|---|---|---|---|
| Taxon | Mean | SD | Mean | SD | p-value | Ratio | AUC | sensitivity | specificity |
| g_Rhizobium | 0.0056 | 0.0056 | 0.0000 | 0.0000 | 0.0000 | 0.00 | 1.00 | 1.00 | 1.00 |
| g_Proteus | 0.0132 | 0.0192 | 0.0006 | 0.0010 | 0.0003 | 0.04 | 0.87 | 0.76 | 0.95 |
| g_Morganella | 0.0211 | 0.0295 | 0.0013 | 0.0060 | 0.0002 | 0.06 | 0.84 | 0.61 | 0.84 |
| g_Acinetobacter | 0.1683 | 0.1396 | 0.0172 | 0.0138 | 0.0000 | 0.10 | 0.94 | 0.84 | 0.95 |
| g_Pseudomonas | 0.1288 | 0.0965 | 0.0286 | 0.0201 | 0.0000 | 0.22 | 0.90 | 0.82 | 0.89 |
| g_SMB53 | 0.0034 | 0.0046 | 0.0008 | 0.0012 | 0.0017 | 0.23 | 0.73 | 0.58 | 0.66 |
| g_Enterococcus | 0.0086 | 0.0108 | 0.0023 | 0.0024 | 0.0010 | 0.26 | 0.74 | 0.50 | 0.82 |
| g_Lactococcus | 0.0035 | 0.0039 | 0.0015 | 0.0019 | 0.0074 | 0.44 | 0.67 | 0.50 | 0.66 |
| g_Turicibacter | 0.0017 | 0.0026 | 0.0038 | 0.0040 | 0.0075 | 2.27 | 0.72 | 0.71 | 0.61 |
| g_Coprococcus | 0.0036 | 0.0043 | 0.0083 | 0.0052 | 0.0000 | 2.31 | 0.81 | 0.74 | 0.76 |
| g_Bacteroides | 0.0212 | 0.0207 | 0.0517 | 0.0381 | 0.0001 | 2.44 | 0.84 | 0.82 | 0.74 |
| g_Dorea | 0.0010 | 0.0015 | 0.0025 | 0.0024 | 0.0011 | 2.55 | 0.80 | 0.79 | 0.66 |
| g_Streptococcus | 0.0143 | 0.0122 | 0.0366 | 0.0166 | 0.0000 | 2.56 | 0.90 | 0.79 | 0.87 |
| g_Lachnospira | 0.0005 | 0.0012 | 0.0017 | 0.0020 | 0.0020 | 3.54 | 0.76 | 0.82 | 0.66 |
| g_[Ruminococcus] | 0.0010 | 0.0015 | 0.0040 | 0.0031 | 0.0000 | 3.83 | 0.85 | 0.79 | 0.58 |
| g_Corynebacterium | 0.0090 | 0.0097 | 0.0366 | 0.0197 | 0.0000 | 4.07 | 0.96 | 0.89 | 0.87 |
| g_Comamonas | 0.0003 | 0.0010 | 0.0013 | 0.0018 | 0.0050 | 4.32 | 0.77 | 0.87 | 0.61 |
| g_Ruminococcus | 0.0030 | 0.0043 | 0.0148 | 0.0073 | 0.0000 | 4.94 | 0.94 | 0.87 | 0.82 |
| g_Gordonia | 0.0001 | 0.0005 | 0.0004 | 0.0006 | 0.0081 | 5.03 | 0.78 | 0.76 | 0.68 |
| g_Paraprevotella | 0.0002 | 0.0007 | 0.0010 | 0.0014 | 0.0015 | 5.70 | 0.80 | 0.89 | 0.66 |
| g_Mycobacterium | 0.0002 | 0.0008 | 0.0009 | 0.0015 | 0.0090 | 5.82 | 0.76 | 0.92 | 0.58 |
| g_Roseburia | 0.0004 | 0.0017 | 0.0032 | 0.0028 | 0.0000 | 8.45 | 0.93 | 0.92 | 0.79 |
| g_Dialister | 0.0005 | 0.0012 | 0.0065 | 0.0046 | 0.0000 | 13.84 | 0.94 | 0.92 | 0.87 |
| g_Slackia | 0.0000 | 0.0003 | 0.0007 | 0.0014 | 0.0062 | 15.14 | 0.77 | 0.92 | 0.58 |
| g_Escherichia | 0.0001 | 0.0001 | 0.0009 | 0.0008 | 0.0000 | 15.98 | 0.95 | 0.95 | 0.84 |
| g_Phascolarctobacterium | 0.0001 | 0.0007 | 0.0026 | 0.0030 | 0.0000 | 20.76 | 0.93 | 0.97 | 0.84 |
| g_Sutterella | 0.0001 | 0.0003 | 0.0013 | 0.0015 | 0.0000 | 20.99 | 0.86 | 0.95 | 0.74 |
| g_Virgibacillus | 0.0000 | 0.0001 | 0.0008 | 0.0010 | 0.0000 | 21.74 | 0.83 | 0.89 | 0.61 |
| g_Eggerthella | 0.0000 | 0.0001 | 0.0005 | 0.0010 | 0.0044 | 24.82 | 0.76 | 0.76 | 0.55 |
| g_Halomonas | 0.0001 | 0.0003 | 0.0017 | 0.0022 | 0.0000 | 26.50 | 0.91 | 0.95 | 0.76 |
| g_Citrobacter | 0.0003 | 0.0005 | 0.0089 | 0.0056 | 0.0000 | 30.07 | 0.99 | 0.97 | 0.92 |
| g_Roseomonas | 0.0000 | 0.0002 | 0.0012 | 0.0016 | 0.0001 | 35.67 | 0.85 | 0.97 | 0.58 |
| g_Alloiococcus | 0.0000 | 0.0001 | 0.0022 | 0.0050 | 0.0097 | 174.52 | 0.88 | 0.95 | 0.74 |
| g_Serratia | 0.0000 | 0.0000 | 0.0006 | 0.0010 | 0.0007 | 195.10 | 0.81 | 0.92 | 0.55 |
| g_Methanobrevibacter | 0.0000 | 0.0000 | 0.0020 | 0.0021 | 0.0000 | 555.27 | 0.92 | 1.00 | 0.74 |
| g_Bilophila | 0.0000 | 0.0000 | 0.0005 | 0.0008 | 0.0003 | 873.83 | 0.79 | 0.92 | 0.53 |

### Example 6. Colon Cancer Diagnostic Model Based on metagenomic Analysis of Bacteria-Derived EVs Isolated from Stool of Colon Polyp Patients and Colon Cancer Patients

Extracellular vesicles were isolated from stool samples of 29 colon cancer patients and 27 colon polyp patients, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

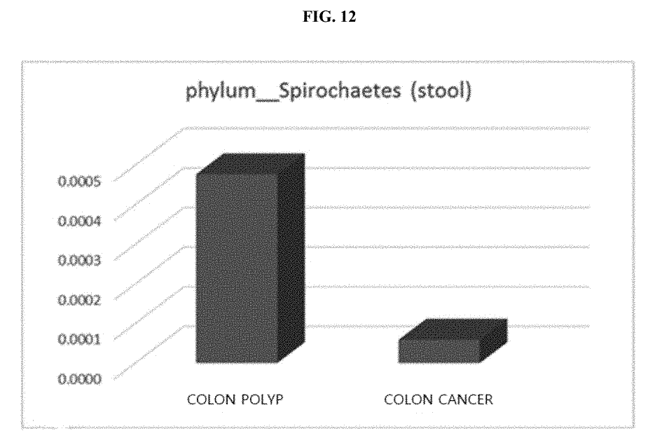

As a result of analyzing bacteria-derived extracellular vesicles in stool at a phylum level, a diagnostic model developed using bacteria belonging to the phylum Spirochaetes as a biomarker exhibited significant diagnostic performance for colon cancer (see Table 12 and FIG. 12).

TABLE 12

| | Colon polyp | | Colon Cancer | | t-test | | | | |
|---|---|---|---|---|---|---|---|---|---|
| Taxon | Mean | SD | Mean | SD | p-value | Ratio | AUC | sensitivity | specificity |
| p_Spirochaetes | 0.0005 | 0.0009 | 0.0001 | 0.0001 | 0.0216 | 0.12 | 0.87 | 0.74 | 0.79 |

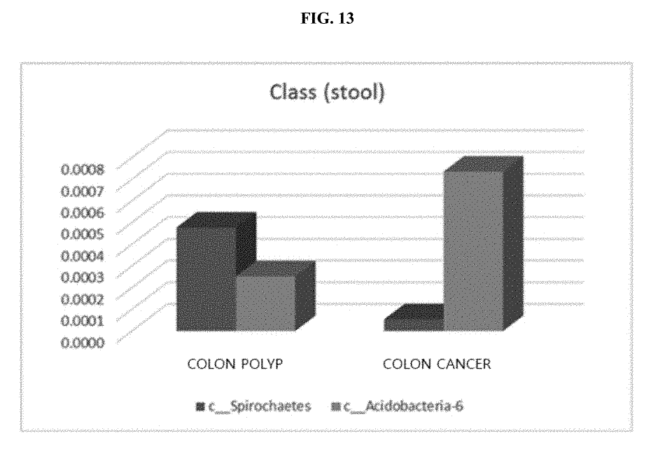

As a result of analyzing bacteria-derived extracellular vesicles in stool at a class level, a diagnostic model developed using bacteria belonging to the class Spirochaetes and the class Acidobacteria-6 as a biomarker exhibited significant diagnostic performance for colon cancer (see Table 13 and FIG. 13).

TABLE 13

| | Colon polyp | | Colon Cancer | | t-test | | | | |
|---|---|---|---|---|---|---|---|---|---|
| Taxon | Mean | SD | Mean | SD | p-value | Ratio | AUC | sensitivity | specificity |
| c_Spirochaetes | 0.0005 | 0.0009 | 0.0001 | 0.0001 | 0.0202 | 0.11 | 0.87 | 0.78 | 0.83 |
| c_Acidobacteria-6 | 0.0003 | 0.0004 | 0.0007 | 0.0010 | 0.0237 | 2.92 | 0.85 | 0.81 | 0.69 |

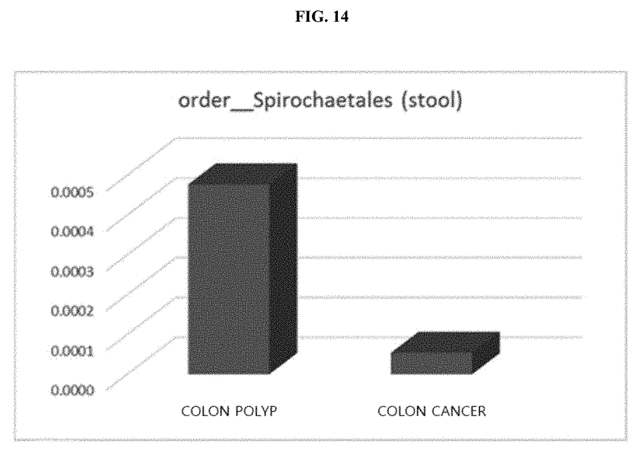

As a result of analyzing bacteria-derived extracellular vesicles in stool at an order level, a diagnostic model developed using bacteria belonging to the order Spirochaetales as a biomarker exhibited significant diagnostic performance for colon cancer (see Table 14 and FIG. 14).

TABLE 14

| | Colon polyp | | Colon Cancer | | t-test | | | | |
|---|---|---|---|---|---|---|---|---|---|
| Taxon | Mean | SD | Mean | SD | p-value | Ratio | AUC | sensitivity | specificity |
| o_Spirochaetales | 0.0005 | 0.0009 | 0.0001 | 0.0001 | 0.0202 | 0.11 | 0.87 | 0.78 | 0.83 |

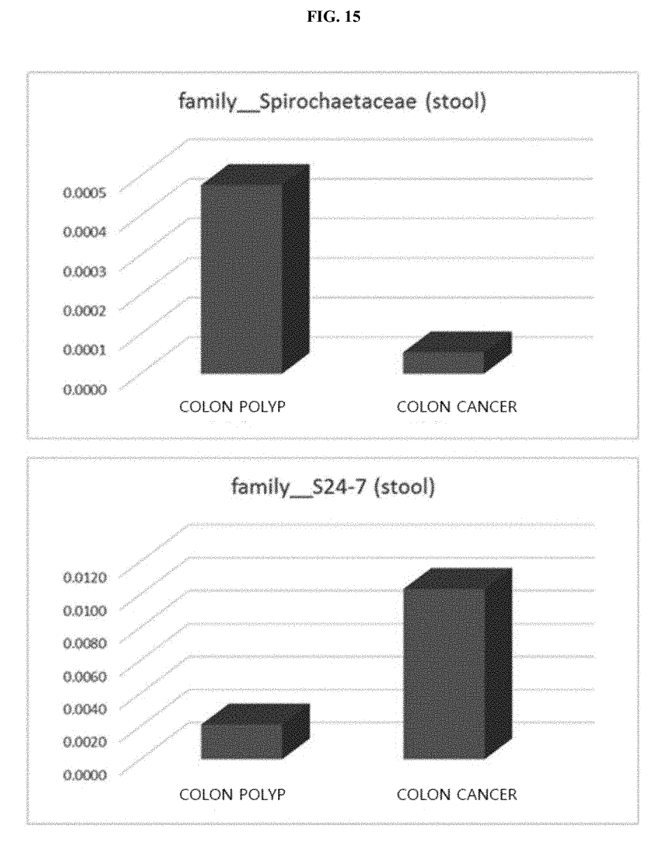

As a result of analyzing bacteria-derived extracellular vesicles in stool at a family level, a diagnostic model developed using bacteria belonging to the family Spirochaetaceae and the family S24-7 as a biomarker exhibited significant diagnostic performance for colon cancer (see Table 15 and FIG. 15).

TABLE 15

| | Colon polyp | | Colon Cancer | | t-test | | | | |
|---|---|---|---|---|---|---|---|---|---|
| Taxon | Mean | SD | Mean | SD | p-value | Ratio | AUC | sensitivity | specificity |
| f_Spirochaetaceae | 0.0005 | 0.0009 | 0.0001 | 0.0001 | 0.0202 | 0.11 | 0.87 | 0.78 | 0.83 |
| f_S24-7 | 0.0021 | 0.0053 | 0.0103 | 0.0206 | 0.0492 | 4.93 | 0.84 | 0.81 | 0.69 |

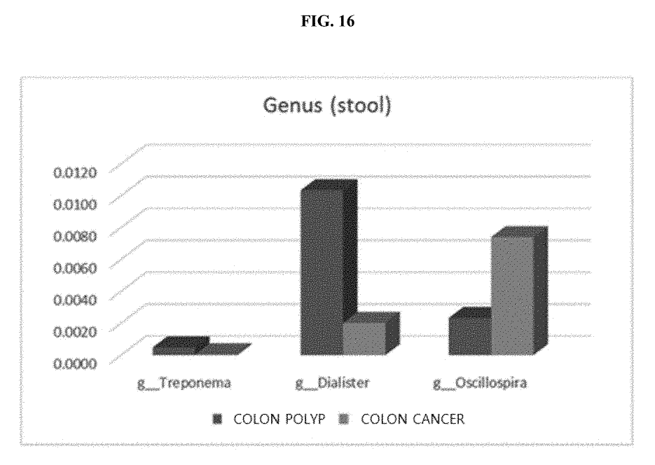

As a result of analyzing bacteria-derived extracellular vesicles in stool at a genus level, a diagnostic model developed using bacteria belonging to the genus Treponema, the genus Dialister, and the genus Oscillospira as a biomarker exhibited significant diagnostic performance for colon cancer (see Table 16 and FIG. 16).

TABLE 16

| | Colon polyp | | Colon Cancer | | t-test | | | | |
|---|---|---|---|---|---|---|---|---|---|
| Taxon | Mean | SD | Mean | SD | p-value | Ratio | AUC | sensitivity | specificity |
| g_Treponema | 0.0005 | 0.0009 | 0.0001 | 0.0001 | 0.0202 | 0.11 | 0.8685 | 0.7778 | 0.8276 |
| g_Dialister | 0.0103 | 0.0152 | 0.0020 | 0.0027 | 0.0106 | 0.20 | 0.8672 | 0.7778 | 0.8276 |
| g_Oscillospira | 0.0023 | 0.0019 | 0.0074 | 0.0109 | 0.0212 | 3.18 | 0.8378 | 0.7778 | 0.7241 |

### Example 7. Colon Cancer Diagnostic Model Based on Metagenomic Analysis of Bacteria-Derived EVs Isolated from Urine of Colon Polyp Patients and Colon Cancer Patients

Extracellular vesicles were isolated from urine samples of 26 colon cancer patients and 38 colon polyp patients, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

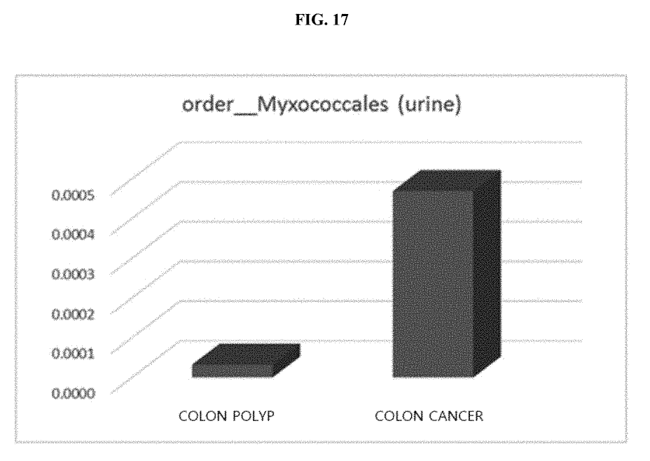

As a result of analyzing bacteria-derived extracellular vesicles in urine at an order level, a diagnostic model developed using bacteria belonging to the order Myxococcales exhibited significant diagnostic performance for colon cancer (see Table 17 and FIG. 17).

TABLE 17

| | Colon polyp | | Colon Cancer | | t-test | | | | |
|---|---|---|---|---|---|---|---|---|---|
| Taxon | Mean | SD | Mean | SD | p-value | Ratio | AUC | sensitivity | specificity |
| o_Myxococcales | 0.0000 | 0.0001 | 0.0005 | 0.0013 | 0.0442 | 14.52 | 0.78 | 0.69 | 0.82 |

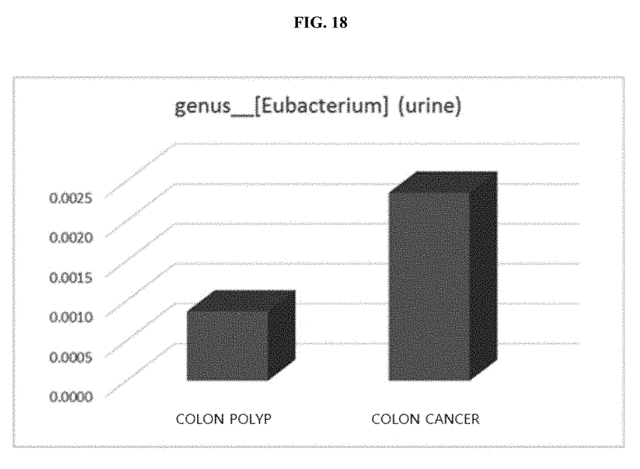

As a result of analyzing bacteria-derived extracellular vesicles in urine at a genus level, a diagnostic model developed using bacteria belonging to the genus Eubacterium exhibited significant diagnostic performance for colon cancer (see Table 18 and FIG. 18).

TABLE 18

| | Colon polyp | | Colon Cancer | | t-test | | | | |
|---|---|---|---|---|---|---|---|---|---|
| Taxon | Mean | SD | Mean | SD | p-value | Ratio | AUC | sensitivity | specificity |
| g_[Eubacterium] | 0.0009 | 0.0011 | 0.0024 | 0.0028 | 0.0040 | 2.72 | 0.79 | 0.62 | 0.79 |

### Example 8. Colon Polyp Diagnostic Model Based on Metagenomic Analysis of Bacteria-Derived EVs Isolated from Stool of Normal Individuals and Colon Polyp Patients

Extracellular vesicles were isolated from stool samples of 27 colon cancer patients and 358 normal individuals, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

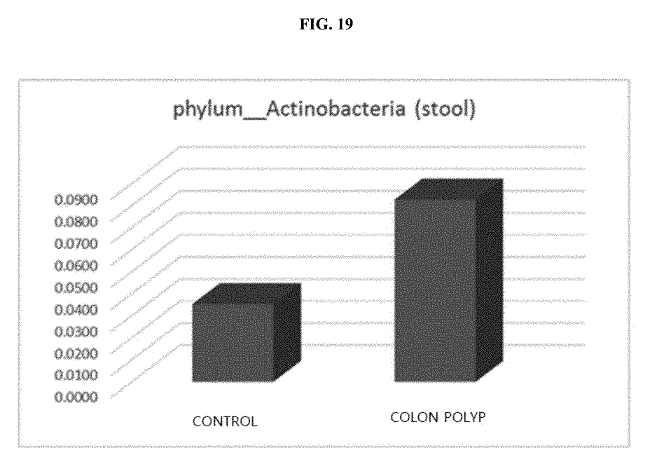

As a result of analyzing bacteria-derived extracellular vesicles in stool at a phylum level, a diagnostic model developed using bacteria belonging to the phylum Actinobacteria exhibited significant diagnostic performance for colon polyp (see Table 19 and FIG. 19).

TABLE 19

| | Control | | Colon polyp | | t-test | | | | |
|---|---|---|---|---|---|---|---|---|---|
| Taxon | Mean | SD | Mean | SD | p-value | Ratio | AUC | sensitivity | specificity |
| p_Actinobacteria | 0.0353 | 0.0403 | 0.0826 | 0.0680 | 0.0016 | 2.34 | 0.84 | 0.99 | 0.11 |

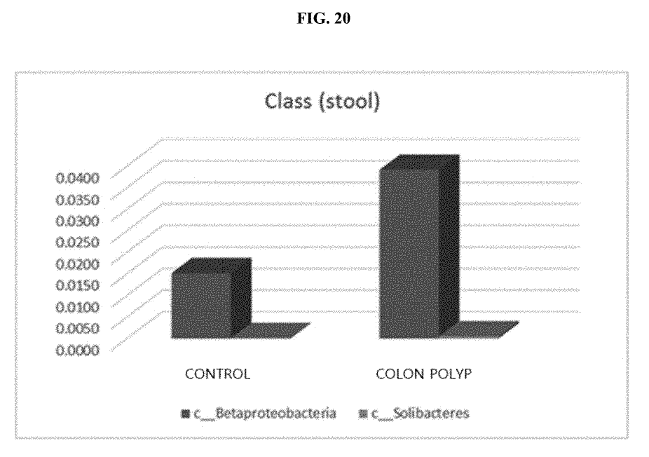

As a result of analyzing bacteria-derived extracellular vesicles in stool at a class level, a diagnostic model developed using bacteria belonging to the class Betaproteobacteria and the class Solibacteres exhibited significant diagnostic performance for colon polyp (see Table 20 and FIG. 20).

TABLE 20

| | Control | | Colon polyp | | t-test | | | | |
|---|---|---|---|---|---|---|---|---|---|
| Taxon | Mean | SD | Mean | SD | p-value | Ratio | AUC | sensitivity | specificity |
| c_Betaproteobacteria | 0.0151 | 0.0370 | 0.0391 | 0.0369 | 0.0013 | 2.58 | 0.82 | 1.00 | 0.00 |
| c_Solibacteres | 0.0001 | 0.0006 | 0.0005 | 0.0007 | 0.0093 | 4.95 | 0.83 | 1.00 | 0.00 |

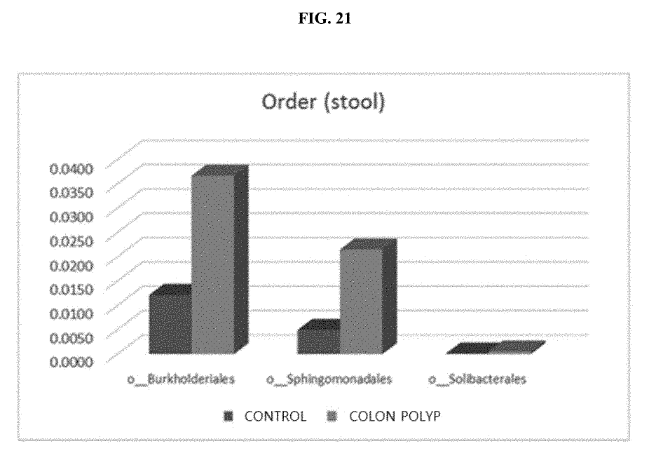

As a result of analyzing bacteria-derived extracellular vesicles in stool at an order level, a diagnostic model developed using bacteria belonging to the order Burkholderiales, the order Sphingomonadales, and the order Solibacterales exhibited significant diagnostic performance for colon polyp (see Table 21 and FIG. 21).

TABLE 21

| | Control | | Colon polyp | | t-test | | | | |
|---|---|---|---|---|---|---|---|---|---|
| Taxon | Mean | SD | Mean | SD | p-value | Ratio | AUC | sensitivity | specificity |
| o_Burkholderiales | 0.0121 | 0.0358 | 0.0367 | 0.0365 | 0.0007 | 3.02 | 0.82 | 1.00 | 0.00 |
| o_Sphingomonadales | 0.0050 | 0.0097 | 0.0215 | 0.0216 | 0.0006 | 4.33 | 0.87 | 0.99 | 0.19 |
| o_Solibacterales | 0.0001 | 0.0006 | 0.0005 | 0.0007 | 0.0091 | 4.98 | 0.83 | 1.00 | 0.00 |

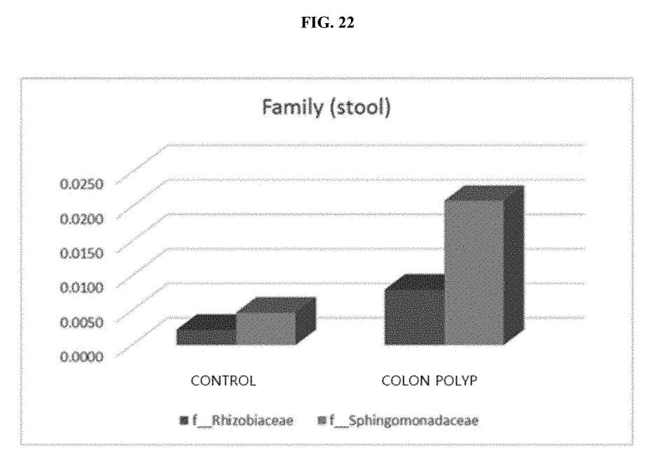

As a result of analyzing bacteria-derived extracellular vesicles in stool at a family level, a diagnostic model developed using bacteria belonging to the family Rhizobiaceae and the family Sphingomonadaceae exhibited significant diagnostic performance for colon polyp (see Table 22 and FIG. 22).

TABLE 22

| | Control | | Colon polyp | | t-test | | | | |
|---|---|---|---|---|---|---|---|---|---|
| Taxon | Mean | SD | Mean | SD | p-value | Ratio | AUC | sensitivity | specificity |
| f_Rhizobiaceae | 0.0022 | 0.0040 | 0.0080 | 0.0105 | 0.0091 | 3.64 | 0.83 | 0.99 | 0.19 |
| f_Sphingomonadaceae | 0.0047 | 0.0091 | 0.0208 | 0.0210 | 0.0006 | 4.46 | 0.87 | 0.99 | 0.22 |

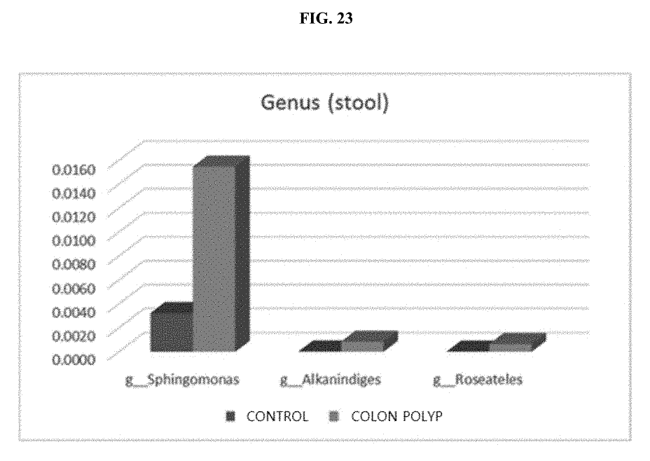

As a result of analyzing bacteria-derived extracellular vesicles in stool at a genus level, a diagnostic model developed using bacteria belonging to the genus Sphingomonas, the genus Alkanindiges, and the genus Roseateles exhibited significant diagnostic performance for colon polyp (see Table 23 and FIG. 23).

TABLE 23

| | Control | | Colon polyp | | t-test | | | | |
|---|---|---|---|---|---|---|---|---|---|
| Taxon | Mean | SD | Mean | SD | p-value | Ratio | AUC | sensitivity | specificity |
| g_Sphingomonas | 0.0032 | 0.0059 | 0.0155 | 0.0164 | 0.0008 | 4.77 | 0.87 | 0.99 | 0.30 |
| g_Alkanindiges | 0.0000 | 0.0002 | 0.0008 | 0.0015 | 0.0097 | 26.73 | 0.84 | 1.00 | 0.26 |
| g_Roseateles | 0.0000 | 0.0002 | 0.0006 | 0.0011 | 0.0078 | 30.88 | 0.85 | 0.99 | 0.22 |

### Example 9. Colon Polyp Diagnostic Model Based on metagenomic Analysis of Bacteria-Derived EVs Isolated from Urine of Normal Individuals and Colon Polyp Patients

Extracellular vesicles were isolated from urine samples of 38 colon cancer patients and 38 normal individuals, and then metagenomic sequencing was performed thereon using the method of Example 3. For the development of a diagnostic model, first, a strain exhibiting a p value of less than 0.05 between two groups in a t-test and a difference of two-fold or more between two groups was selected, and then an area under curve (AUC), sensitivity, and specificity, which are diagnostic performance indexes, were calculated by logistic regression analysis.

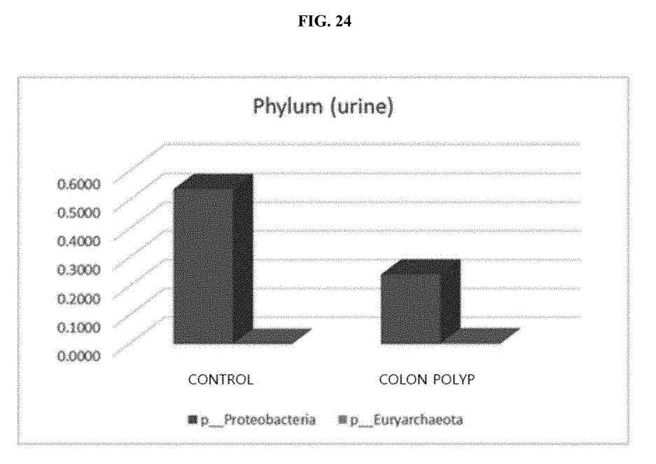

As a result of analyzing bacteria-derived extracellular vesicles in urine at a phylum level, a diagnostic model developed using bacteria belonging to the phylum Proteobacteria and the phylum Euryarchaeota exhibited significant diagnostic performance for colon polyp (see Table 24 and FIG. 24).

TABLE 24

| | Control | | Colon polyp | | t-test | | | | |
|---|---|---|---|---|---|---|---|---|---|
| Taxon | Mean | SD | Mean | SD | p-value | Ratio | AUC | sensitivity | specificity |
| p_Proteobacteria | 0.5379 | 0.1871 | 0.2416 | 0.0691 | 0.0000 | 0.45 | 0.92 | 0.82 | 0.88 |
| p_Euryarchaeota | 0.0000 | 0.0001 | 0.0024 | 0.0027 | 0.0001 | 82.21 | 0.90 | 1.00 | 0.73 |

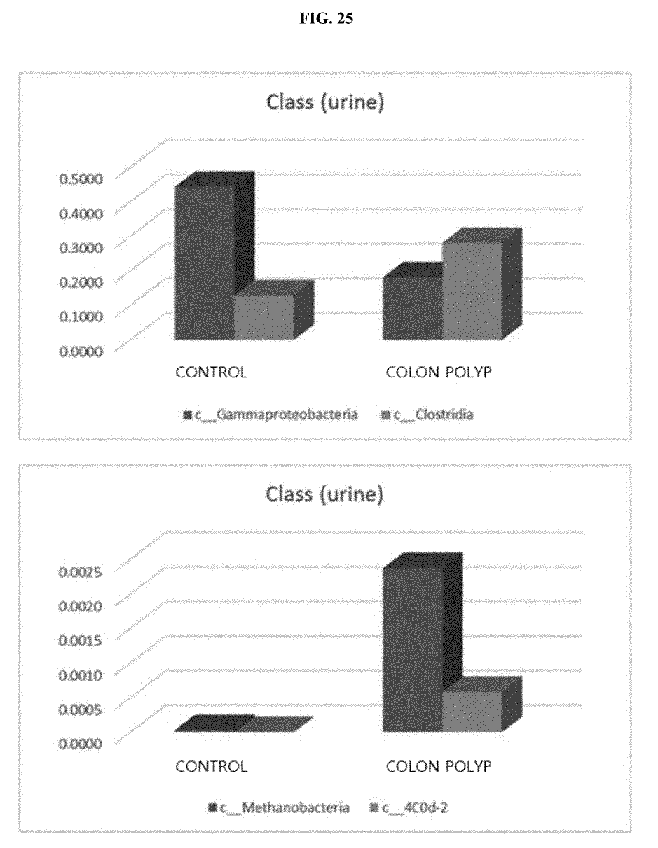

As a result of analyzing bacteria-derived extracellular vesicles in urine at a class level, a diagnostic model developed using bacteria belonging to the class Gammaproteobacteria, the class Clostridia, the class Methanobacteria, and the class 4C0d-2 exhibited significant diagnostic performance for colon polyp (see Table 25 and FIG. 25).

TABLE 25

| | Control | | Colon polyp | | t-test | | | | |
|---|---|---|---|---|---|---|---|---|---|
| Taxon | Mean | SD | Mean | SD | p-value | Ratio | AUC | sensitivity | specificity |
| c_Gammaproteobacteria | 0.4431 | 0.2104 | 0.1807 | 0.0511 | 0.0000 | 0.41 | 0.86 | 0.76 | 0.88 |
| c_Clostridin | 0.1286 | 0.0723 | 0.2804 | 0.0637 | 0.0000 | 2.18 | 0.94 | 0.87 | 0.77 |
| c_Methanobacteria | 0.0000 | 0.0001 | 0.0024 | 0.0027 | 0.0002 | 79.88 | 0.90 | 1.00 | 0.73 |
| c_4C0d-2 | 0.0000 | 0.0000 | 0.0006 | 0.0011 | 0.0099 | 1952.62 | 0.81 | 0.92 | 0.38 |

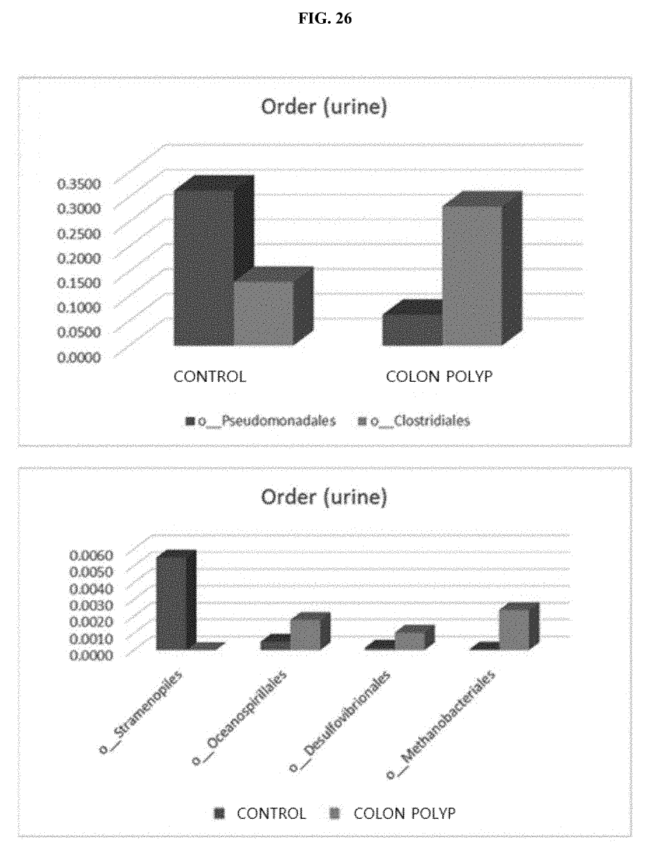

As a result of analyzing bacteria-derived extracellular vesicles in urine at an order level, a diagnostic model developed using bacteria belonging to the order Stramenopiles, the order Pseudomonadales, the order Clostridiales, the order Oceanospirillales, the order Desulfovibrionales, and the order Methanobacteriales exhibited significant diagnostic performance for colon polyp (see Table 26 and FIG. 26).

TABLE 26

| | Control | | Colon polyp | | t-test | | | | |
|---|---|---|---|---|---|---|---|---|---|
| Taxon | Mean | SD | Mean | SD | p-value | Ratio | Auc | sensitivity | specificity |
| o_Stramenopiles | 0.0055 | 0.0083 | 0.0000 | 0.0000 | 0.0014 | 0.00 | 0.89 | 0.74 | 1.00 |
| o_Pseudomonadales | 0.3124 | 0.1910 | 0.0622 | 0.0414 | 0.0000 | 0.20 | 0.91 | 0.84 | 0.85 |
| o_Clostridiales | 0.1284 | 0.0725 | 0.2804 | 0.0637 | 0.0000 | 2.18 | 0.94 | 0.87 | 0.77 |
| o_Oceanospirillales | 0.0005 | 0.0012 | 0.0018 | 0.0015 | 0.0003 | 3.65 | 0.86 | 0.89 | 0.62 |
| o_Desulfovibrionales | 0.0001 | 0.0005 | 0.0010 | 0.0015 | 0.0064 | 7.61 | 0.83 | 0.92 | 0.54 |
| o_Methanobacteriales | 0.0000 | 0.0001 | 0.0024 | 0.0027 | 0.0002 | 79.88 | 0.90 | 1.00 | 0.73 |

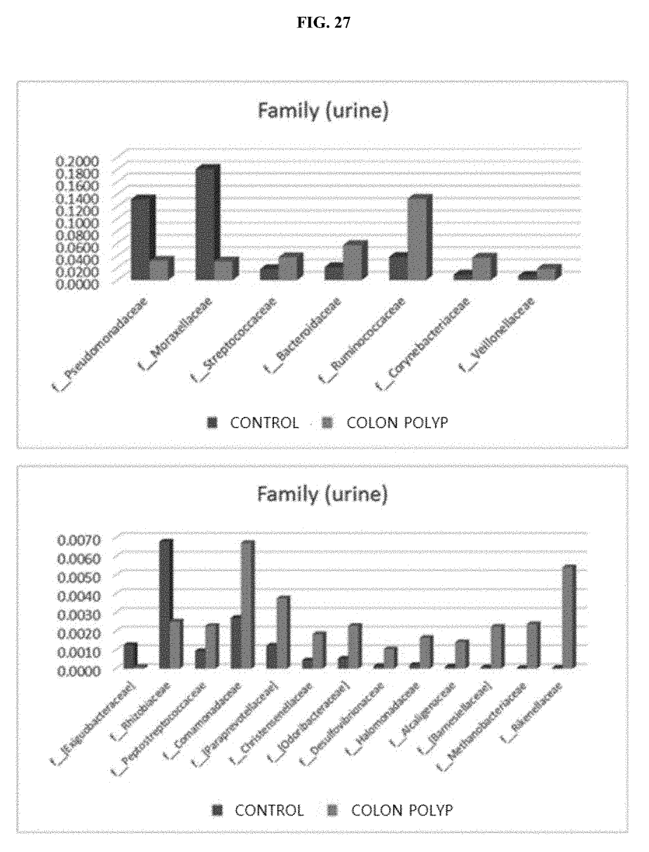

As a result of analyzing bacteria-derived extracellular vesicles in urine at a family level, a diagnostic model developed using bacteria belonging to the family Exiguobacteraceae, the family Moraxellaceae, the family Pseudomonadaceae, the family Rhizobiaceae, the family Streptococcaceae, the family Peptostreptococcaceae, the family Comamonadaceae, the family Veillonellaceae, the family Bacteroidaceae, the family Paraprevotellaceae, the family Ruminococcaceae, the family Corynebacteriaceae, the family Christensenellaceae, the family Odoribacteraceae, the family Desulfovibrionaceae, the family Halomonadaceae, the family Alcaligenaceae, the family Barnesiellaceae, the family Methanobacteriaceae, and the family Rikenellaceae exhibited significant diagnostic performance for colon polyp (see Table 27 and FIG. 27).

TABLE 27

| | Control | | Colon polyp | | t-test | | | | |
|---|---|---|---|---|---|---|---|---|---|
| Taxon | Mean | SD | Mean | SD | p-value | Ratio | AUC | sensitivity | specificity |
| f_(Exiguobacteraceae) | 0.0013 | 0.0026 | 0.0001 | 0.0003 | 0.0076 | 0.06 | 0.74 | 0.63 | 0.77 |
| f_Moraxellaceae | 0.1810 | 0.1401 | 0.0306 | 0.0235 | 0.0000 | 0.17 | 0.88 | 0.79 | 0.81 |
| f_Pseudomonadaceae | 0.1314 | 0.0973 | 0.0315 | 0.0201 | 0.0000 | 0.24 | 0.89 | 0.82 | 0.85 |
| f_Rhizobiaceae | 0.0068 | 0.0062 | 0.0025 | 0.0032 | 0.0007 | 0.37 | 0.83 | 0.79 | 0.62 |
| f_Streptococcaceae | 0.0180 | 0.0126 | 0.0373 | 0.0099 | 0.0000 | 2.08 | 0.90 | 0.87 | 0.85 |
| f_Peptostreptococcaceae | 0.0009 | 0.0017 | 0.0023 | 0.0017 | 0.0029 | 2.42 | 0.84 | 0.95 | 0.65 |
| f_Comamonadaceae | 0.0027 | 0.0036 | 0.0067 | 0.0067 | 0.0090 | 2.48 | 0.77 | 0.79 | 0.46 |
| f_Veillonellaceae | 0.0070 | 0.0083 | 0.0186 | 0.0056 | 0.0000 | 2.67 | 0.86 | 0.79 | 0.77 |
| f_Bacteroidaceae | 0.0212 | 0.0207 | 0.0571 | 0.0217 | 0.0000 | 2.69 | 0.92 | 0.89 | 0.73 |
| f_[Paraprevotellaceae] | 0.0012 | 0.0036 | 0.0037 | 0.0021 | 0.0008 | 3.07 | 0.85 | 0.82 | 0.65 |
| f_Ruminococcaceae | 0.0379 | 0.0431 | 0.1325 | 0.0396 | 0.0000 | 3.50 | 0.93 | 0.87 | 0.85 |
| f_Corynebacteriaceae | 0.0090 | 0.0097 | 0.0367 | 0.0159 | 0.0000 | 4.09 | 0.97 | 0.95 | 0.85 |
| f_Christensenellaceae | 0.0004 | 0.0013 | 0.0018 | 0.0021 | 0.0042 | 4.23 | 0.84 | 0.92 | 0.62 |
| f_[Odoribacteraceae] | 0.0005 | 0.0022 | 0.0023 | 0.0022 | 0.0026 | 4.30 | 0.83 | 0.89 | 0.54 |
| f_Desulfovibrionaceae | 0.0001 | 0.0005 | 0.0010 | 0.0015 | 0.0064 | 7.61 | 0.83 | 0.92 | 0.54 |
| f_Halomonadaceae | 0.0002 | 0.0008 | 0.0016 | 0.0015 | 0.0001 | 8.48 | 0.91 | 0.97 | 0.73 |
| f_Alcaligenaceae | 0.0001 | 0.0004 | 0.0014 | 0.0017 | 0.0006 | 12.38 | 0.82 | 0.92 | 0.50 |
| f_[Barnesiellaceae] | 0.0001 | 0.0004 | 0.0022 | 0.0031 | 0.0015 | 33.09 | 0.93 | 0.97 | 0.77 |
| f_Methanobacteriaceae | 0.0000 | 0.0001 | 0.0024 | 0.0027 | 0.0002 | 79.88 | 0.90 | 1.00 | 0.73 |
| f_Rikenellaceae | 0.0000 | 0.0002 | 0.0054 | 0.0033 | 0.0000 | 139.26 | 0.97 | 0.97 | 0.92 |

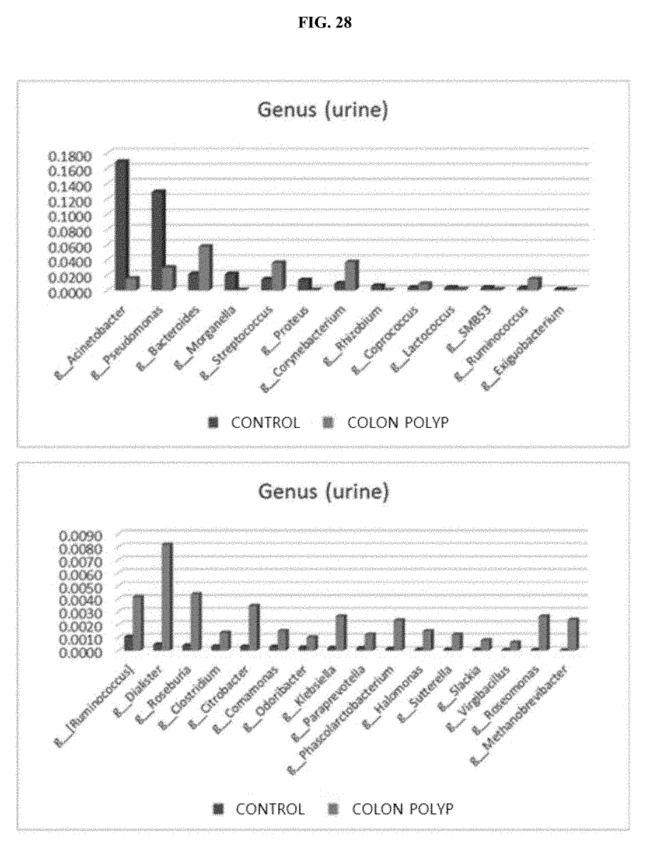

As a result of analyzing bacteria-derived extracellular vesicles in urine at a genus level, a diagnostic model developed using bacteria belonging to the genus Rhizobium, the genus Morganella, the genus Proteus, the genus Exiguobacterium, the genus Acinetobacter, the genus Pseudomonas, the genus SMB53, the genus Lactococcus, the genus Coprococcus, the genus Streptococcus, the genus Bacteroides, the genus Ruminococcus, the genus Corynebacterium, the genus Odoribacter, the genus Clostridium, the genus Comamonas, the genus Paraprevotella, the genus Roseburia, the genus Citrobacter, the genus Klebsiella, the genus Virgibacillus, the genus Slackia, the genus Dialister, the genus Phascolarctobacterium, the genus Sutterella, the genus Halomonas, the genus Roseomonas, and the genus Methanobrevibacter exhibited significant diagnostic performance for colon polyp (see Table 28 and FIG. 28).

TABLE 28

| | Control | | Colon polyp | | t-test | | | | |
|---|---|---|---|---|---|---|---|---|---|
| Taxon | Mean | SD | Mean | SD | p-value | Ratio | AUC | sensitivity | specificity |
| g_Rhizobium | 0.0056 | 0.0056 | 0.0000 | 0.0000 | 0.0000 | 0.00 | 0.99 | 0.95 | 1.00 |
| g_Morganella | 0.0211 | 0.0295 | 0.0003 | 0.0006 | 0.0001 | 0.01 | 0.82 | 0.63 | 0.85 |
| g_Proteus | 0.0132 | 0.0192 | 0.0003 | 0.0005 | 0.0002 | 0.02 | 0.91 | 0.82 | 0.96 |
| g_Exiguobacterium | 0.0013 | 0.0026 | 0.0001 | 0.0003 | 0.0078 | 0.06 | 0.74 | 0.63 | 0.77 |
| g_Acinetobacter | 0.1683 | 0.1396 | 0.0152 | 0.0123 | 0.0000 | 0.09 | 0.90 | 0.82 | 0.81 |
| g_Pseudomonas | 0.1288 | 0.0965 | 0.0296 | 0.0195 | 0.0000 | 0.23 | 0.89 | 0.82 | 0.81 |
| g_SMB53 | 0.0034 | 0.0046 | 0.0010 | 0.0010 | 0.0039 | 0.30 | 0.78 | 0.74 | 0.69 |
| g_Lactococcus | 0.0035 | 0.0039 | 0.0014 | 0.0014 | 0.0029 | 0.39 | 0.75 | 0.71 | 0.62 |
| g_Coprococcus | 0.0036 | 0.0043 | 0.0084 | 0.0047 | 0.0001 | 2.35 | 0.82 | 0.87 | 0.73 |
| g_Streptococcus | 0.0143 | 0.0122 | 0.0358 | 0.0102 | 0.0000 | 2.50 | 0.90 | 0.87 | 0.81 |
| g_Bacteroides | 0.0212 | 0.0207 | 0.0571 | 0.0217 | 0.0000 | 2.69 | 0.92 | 0.89 | 0.73 |
| g_[Ruminococcus] | 0.0010 | 0.0015 | 0.0041 | 0.0039 | 0.0005 | 3.96 | 0.81 | 0.87 | 0.58 |
| g_Corynebacterium | 0.0090 | 0.0097 | 0.0367 | 0.0159 | 0.0000 | 4.09 | 0.97 | 0.95 | 0.85 |
| g_Odoribacter | 0.0002 | 0.0009 | 0.0010 | 0.0012 | 0.0050 | 4.31 | 0.82 | 0.92 | 0.54 |
| g_Clostridium | 0.0003 | 0.0011 | 0.0014 | 0.0015 | 0.0016 | 4.39 | 0.84 | 0.95 | 0.58 |
| g_Ruminococcus | 0.0030 | 0.0043 | 0.0145 | 0.0083 | 0.0000 | 4.84 | 0.91 | 0.89 | 0.73 |
| g_Comamonas | 0.0003 | 0.0010 | 0.0015 | 0.0020 | 0.0087 | 5.17 | 0.78 | 0.92 | 0.50 |
| g_Paraprevotella | 0.0002 | 0.0007 | 0.0012 | 0.0014 | 0.0010 | 6.62 | 0.85 | 0.92 | 0.62 |
| g_Roseburia | 0.0004 | 0.0017 | 0.0043 | 0.0029 | 0.0000 | 11.30 | 0.96 | 0.95 | 0.88 |
| g_Citrobacter | 0.0003 | 0.0005 | 0.0034 | 0.0020 | 0.0000 | 11.16 | 0.97 | 0.95 | 0.92 |
| g_Klebsiella | 0.0002 | 0.0006 | 0.0026 | 0.0042 | 0.0070 | 13.68 | 0.89 | 0.95 | 0.58 |
| g_Virgibacillus | 0.0000 | 0.0001 | 0.0006 | 0.0010 | 0.0074 | 15.87 | 0.75 | 0.92 | 0.42 |
| g_Slackia | 0.0000 | 0.0003 | 0.0008 | 0.0007 | 0.0000 | 16.11 | 0.88 | 0.95 | 0.65 |
| g_Dialister | 0.0005 | 0.0012 | 0.0082 | 0.0047 | 0.0000 | 17.52 | 0.95 | 0.97 | 0.85 |
| g_Phascolarctobacterium | 0.0001 | 0.0007 | 0.0023 | 0.0025 | 0.0002 | 18.69 | 0.88 | 0.97 | 0.65 |
| g_Sutterella | 0.0001 | 0.0003 | 0.0012 | 0.0017 | 0.0015 | 19.16 | 0.80 | 0.97 | 0.46 |
| g_Halomonas | 0.0001 | 0.0003 | 0.0015 | 0.0015 | 0.0001 | 22.99 | 0.91 | 0.95 | 0.73 |
| g_Roseomonas | 0.0000 | 0.0002 | 0.0026 | 0.0043 | 0.0052 | 78.36 | 0.93 | 0.97 | 0.77 |
| g_Methanobrevibacter | 0.0000 | 0.0000 | 0.0024 | 0.0027 | 0.0002 | 661.93 | 0.91 | 1.00 | 0.73 |

The above description of the present invention is provided only for illustrative purposes, and it will be understood by one of ordinary skill in the art to which the present invention pertains that the invention may be embodied in various modified forms without departing from the spirit or essential characteristics thereof. Thus, the embodiments described herein should be considered in an illustrative sense only and not for the purpose of limitation.

### INDUSTRIAL APPLICABILITY

A method of diagnosing colon tumors through bacterial metagenomic analysis, according to the present invention, can be used to predict a risk for colon tumors such as a colon polyp, colon cancer, and the like and diagnose colon tumors by analyzing an increase or decrease in content of extracellular vesicles derived from specific bacteria through bacterial metagenomic analysis using a subject-derived sample. Extracellular vesicles secreted from bacteria present in the environment are absorbed into the human body, and thus may directly affect the occurrence of inflammation and cancer, and it is difficult to diagnose a colon polyp and colon cancer early before symptoms occur, and thus effective treatment thereof is difficult. Thus, according to the present invention, a risk for colon tumors such as a colon polyp, colon cancer, and the like can be predicted through metagenomic analysis of bacteria or bacteria-derived extracellular vesicles using a human body-derived sample, and thus the onset of colon tumors can be delayed or colon tumors can be prevented through appropriate management by early diagnosis and prediction of a risk group for a colon tumor, and, even after a colon tumor occurs, early diagnosis for a colon tumor can be implemented, thereby lowering an incidence rate of a colon tumor and increasing therapeutic effects. In addition, patients diagnosed with a colon polyp or colon cancer are able to avoid exposure to causative factors predicted by the bacterial metagenomic analysis according to the present invention, whereby the progression of a colon polyp and colon cancer can be ameliorated, or recurrence thereof can be prevented.

1. A method of providing information for colon tumor diagnosis, the method comprising the following processes:
(a) extracting DNA from extracellular vesicles isolated from a subject sample;
(b) performing polymerase chain reaction (PCR) on the extracted DNA using a pair of primers having SEQ ID NO: 1 and SEQ ID NO: 2; and
(c) comparing an increase or decrease in content of bacteria-derived extracellular vesicles between a normal individual-derived sample and a colon cancer patient-derived sample through sequencing of a product of the PCR,
comparing an increase or decrease in content of bacteria-derived extracellular vesicles between a colon polyp patient-derived sample and a colon cancer patient-derived sample through sequencing of a product of the PCR, or
comparing an increase or decrease in content of bacteria-derived extracellular vesicles between a normal individual-derived sample and a colon polyp patient-derived sample through sequencing of a product of the PCR.

2. The method of claim 1, wherein, the colon cancer in process (c) is diagnosed by comparing an increase or decrease in content of bacteria-derived extracellular vesicles between a normal individual-derived sample and a colon cancer patient-derived sample through sequencing of a product of the PCR.

3. The method of claim 2, wherein the comparing comprises comparing an increase or decrease in content of:
extracellular vesicles derived from one or more bacteria selected from the group consisting of the phylum Deferribacteres, the phylum Tenericutes, the phylum Actinobacteria, the phylum Acidobacteria, the phylum Armatimonadetes, the phylum Planctomycetes, the phylum Fusobacteria, the phylum Proteobacteria, and the phylum Euryarchaeota;
extracellular vesicles derived from one or more bacteria selected from the group consisting of the class Deferribacteres, the class Mollicutes, the class 4C0d-2, the class Bacilli, the class Alphaproteobacteria, the class Saprospirae, the class Fimbriimonadia, the class Acidobacteria-6, the class Solibacteres, the class Coriobacteriia, the class Oscillatoriophycideae, the class Fusobacteriia, the class Gammaproteobacteria, the class Clostridia, and the class Methanobacteria;
extracellular vesicles derived from one or more bacteria selected from the group consisting of the order RF32, the order YS2, the order Deferribacterales, the order Turicibacterales, the order RF39, the order Oceanospirillales, the order Rhizobiales, the order Lactobacillales, the order Rhodobacterales, the order Saprospirales, the order Sphingomonadales, the order Fimbriimonadales, the order iii1-15, the order Solibacterales, the order Coriobacteriales, the order Chroococcales, the order Fusobacteriales, the order Bdellovibrionales, the order Desulfobacterales, the order Stramenopiles, the order Pseudomonadales, the order Clostridiales, the order Desulfovibrionales, and the order Methanobacteriales;
extracellular vesicles derived from one or more bacteria selected from the group consisting of the family Peptococcaceae, the family Deferribacteraceae, the family Turicibacteraceae, the family Halomonadaceae, the family Clostridiaceae, the family Prevotellaceae, the family Peptostreptococcaceae, the family Rhodobacteraceae, the family Nocardioidaceae, the family Sphingomonadaceae, the family Bartonellaceae, the family Cellulomonadaceae, the family Lactobacillaceae, the family Rhizobiaceae, the family Fimbriimonadaceae, the family Dermacoccaceae, the family Leptotrichiaceae, the family Coriobacteriaceae, the family Xenococcaceae, the family Aeromonadaceae, the family Geodermatophilaceae, the family Bdellovibrionaceae, the family Moraxellaceae, the family Pseudomonadaceae, the family Streptococcaceae, the family Veillonellaceae, the family Bacteroidaceae, the family Aerococcaceae, the family Comamonadaceae, the family Paraprevotellaceae, the family Christensenellaceae, the family Ruminococcaceae, the family Corynebacteriaceae, the family Gordoniaceae, the family Mycobacteriaceae, the family Desulfovibrionaceae, the family Alcaligenaceae, the family Barnesiellaceae, the family Methanobacteriaceae, and the family Rikenellaceae; or
extracellular vesicles derived from one or more bacteria selected from the group consisting of the genus rc4-4, the genus Proteus, the genus Catenibacterium, the genus Mucispirillum, the genus Eubacterium, the genus Turicibacter, the genus Alloiococcus, the genus Halomonas, the genus Prevotella, the genus Dialister, the genus Anaerostipes, the genus SMB53, the genus Faecalibacterium, the genus Blautia, the genus Capnocytophaga, the genus Sphingomonas, the genus Lactobacillus, the genus Fimbriimonas, the genus Dermacoccus, the genus Achromobacter, the genus Novosphingobium, the genus Sneathia, the genus Agrobacterium, the genus Blastomonas, the genus Bdellovibrio, the genus Alkanindiges, the genus Roseateles, the genus Shuttleworthia, the genus Rhizobium, the genus Morganella, the genus Acinetobacter, the genus Pseudomonas, the genus Enterococcus, the genus Lactococcus, the genus Coprococcus, the genus Bacteroides, the genus Dorea, the genus Streptococcus, the genus Lachnospira, the genus Ruminococcus, the genus Corynebacterium, the genus Comamonas, the genus Gordonia, the genus Paraprevotella, the genus Mycobacterium, the genus Roseburia, the genus Slackia, the genus Escherichia, the genus Phascolarctobacterium, the genus Sutterella, the genus Virgibacillus, the genus Eggerthella, the genus Citrobacter, the genus Roseomonas, the genus Serratia, the genus Methanobrevibacter, and the genus Bilophila.

4. The method of claim 1, wherein the colon cancer in process (c) is diagnosed by comparing an increase or decrease in content of bacteria-derived extracellular vesicles between a colon polyp patient-derived sample and a colon cancer patient-derived sample through sequencing of a product of the PCR.

5. The method of claim 4, wherein the comparing comprises comparing an increase or decrease in content of:
extracellular vesicles derived from the bacteria belonging to the phylum Spirochaetes;
extracellular vesicles derived from one or more bacteria selected from the group consisting of the class Spirochaetes and the class Acidobacteria-6;
extracellular vesicles derived from one or more bacteria selected from the group consisting of the order Spirochaetales and the order Myxococcales;
extracellular vesicles derived from one or more bacteria selected from the group consisting of the family Spirochaetaceae and the family S24-7; or
extracellular vesicles derived from one or more bacteria selected from the group consisting of the genus Treponema, the genus Dialister, the genus Oscillospira, and the genus Eubacterium.

6. The method of claim 1, wherein the colon polyp in process (c) is diagnosed by comparing an increase or decrease in content of bacteria-derived extracellular vesicles between a normal individual-derived sample and a colon polyp patient-derived sample through sequencing of a product of the PCR.

7. The method of claim 6, wherein the comparing comprises comparing an increase or decrease in content of:
extracellular vesicles derived from one or more bacteria selected from the group consisting of the phylum Actinobacteria, the phylum Proteobacteria, and the phylum Euryarchaeota;
extracellular vesicles derived from one or more bacteria selected from the group consisting of the class Betaproteobacteria, the class Solibacteres, the class Gammaproteobacteria, the class Clostridia, the class Methanobacteria, and the class 4C0d-2;
extracellular vesicles derived from one or more bacteria selected from the group consisting of the order Burkholderiales, the order Sphingomonadales, the order Solibacterales, the order Stramenopiles, the order Pseudomonadales, the order Clostridiales, the order Oceanospirillales, the order Desulfovibrionales, and the order Methanobacteriales;
extracellular vesicles derived from one or more bacteria selected from the group consisting of the family Rhizobiaceae, the family Sphingomonadaceae, the family Exiguobacteraceae, the family Moraxellaceae, the family Pseudomonadaceae, the family Streptococcaceae, the family Peptostreptococcaceae, the family Comamonadaceae, the family Veillonellaceae, the family Bacteroidaceae, the family Paraprevotellaceae, the family Ruminococcaceae, the family Corynebacteriaceae, the family Christensenellaceae, the family Odoribacteraceae, the family Desulfovibrionaceae, the family Halomonadaceae, the family Alcaligenaceae, the family Barnesiellaceae, the family Methanobacteriaceae, and the family Rikenellaceae; or
extracellular vesicles derived from one or more bacteria selected from the group consisting of the genus Sphingomonas, the genus Alkanindiges, the genus Roseateles, the genus Rhizobium, the genus Morganella, the genus Proteus, the genus Exiguobacterium, the genus Acinetobacter, the genus Pseudomonas, the genus SMB53, the genus Lactococcus, the genus Coprococcus, the genus Streptococcus, the genus Bacteroides, the genus Ruminococcus, the genus Corynebacterium, the genus Odoribacter, the genus Clostridium, the genus Comamonas, the genus Paraprevotella, the genus Roseburia, the genus Citrobacter, the genus Klebsiella, the genus Virgibacillus, the genus Slackia, the genus Dialister, the genus Phascolarctobacterium, the genus Sutterella, the genus Halomonas, the genus Roseomonas, and the genus Methanobrevibacter.

8. The method of claim 1, wherein the subject sample is stool or urine.

9. A method of diagnosing a colon tumor comprising the following processes:
(a) extracting DNA from extracellular vesicles isolated from a subject sample;
(b) performing polymerase chain reaction (PCR) on the extracted DNA using a pair of primers having SEQ ID NO: 1 and SEQ ID NO: 2; and
(c) comparing an increase or decrease in content of bacteria-derived extracellular vesicles between a normal individual-derived sample and a colon cancer patient-derived sample through sequencing of a product of the PCR,
comparing an increase or decrease in content of bacteria-derived extracellular vesicles between a colon polyp patient-derived sample and a colon cancer patient-derived sample through sequencing of a product of the PCR, or
comparing an increase or decrease in content of bacteria-derived extracellular vesicles between a normal individual-derived sample and a colon polyp patient-derived sample through sequencing of a product of the PCR.

10. The method of claim 9, wherein the colon cancer in process (c) is diagnosed by comparing an increase or decrease in content of bacteria-derived extracellular vesicles between a normal individual-derived sample and a colon cancer patient-derived sample through sequencing of a product of the PCR.

11. The method of claim 10, wherein the comparing comprises comparing an increase or decrease in content of:
extracellular vesicles derived from one or more bacteria selected from the group consisting of the phylum Deferribacteres, the phylum Tenericutes, the phylum Actinobacteria, the phylum Acidobacteria, the phylum Armatimonadetes, the phylum Planctomycetes, the phylum Fusobacteria, the phylum Proteobacteria, and the phylum Euryarchaeota;
extracellular vesicles derived from one or more bacteria selected from the group consisting of the class Deferribacteres, the class Mollicutes, the class 4C0d-2, the class Bacilli, the class Alphaproteobacteria, the class Saprospirae, the class Fimbriimonadia, the class Acidobacteria-6, the class Solibacteres, the class Coriobacteriia, the class Oscillatoriophycideae, the class Fusobacteriia, the class Gammaproteobacteria, the class Clostridia, and the class Methanobacteria;
extracellular vesicles derived from one or more bacteria selected from the group consisting of the order RF32, the order YS2, the order Deferribacterales, the order Turicibacterales, the order RF39, the order Oceanospirillales, the order Rhizobiales, the order Lactobacillales, the order Rhodobacterales, the order Saprospirales, the order Sphingomonadales, the order Fimbriimonadales, the order iii1-15, the order Solibacterales, the order Coriobacteriales, the order Chroococcales, the order Fusobacteriales, the order Bdellovibrionales, the order Desulfobacterales, the order Stramenopiles, the order Pseudomonadales, the order Clostridiales, the order Desulfovibrionales, and the order Methanobacteriales;
extracellular vesicles derived from one or more bacteria selected from the group consisting of the family Peptococcaceae, the family Deferribacteraceae, the family Turicibacteraceae, the family Halomonadaceae, the family Clostridiaceae, the family Prevotellaceae, the family Peptostreptococcaceae, the family Rhodobacteraceae, the family Nocardioidaceae, the family Sphingomonadaceae, the family Bartonellaceae, the family Cellulomonadaceae, the family Lactobacillaceae, the family Rhizobiaceae, the family Fimbriimonadaceae, the family Dermacoccaceae, the family Leptotrichiaceae, the family Coriobacteriaceae, the family Xenococcaceae, the family Aeromonadaceae, the family Geodermatophilaceae, the family Bdellovibrionaceae, the family Moraxellaceae, the family Pseudomonadaceae, the family Streptococcaceae, the family Veillonellaceae, the family Bacteroidaceae, the family Aerococcaceae, the family Comamonadaceae, the family Paraprevotellaceae, the family Christensenellaceae, the family Ruminococcaceae, the family Corynebacteriaceae, the family Gordoniaceae, the family Mycobacteriaceae, the family Desulfovibrionaceae, the family Alcaligenaceae, the family Barnesiellaceae, the family Methanobacteriaceae, and the family Rikenellaceae; or
extracellular vesicles derived from one or more bacteria selected from the group consisting of the genus rc4-4, the genus Proteus, the genus Catenibacterium, the genus Mucispirillum, the genus Eubacterium, the genus Turicibacter, the genus Alloiococcus, the genus Halomonas, the genus Prevotella, the genus Dialister, the genus Anaerostipes, the genus SMB53, the genus Faecalibacterium, the genus Blautia, the genus Capnocytophaga, the genus Sphingomonas, the genus Lactobacillus, the genus Fimbriimonas, the genus Dermacoccus, the genus Achromobacter, the genus Novosphingobium, the genus Sneathia, the genus Agrobacterium, the genus Blastomonas, the genus Bdellovibrio, the genus Alkanindiges, the genus Roseateles, the genus Shuttleworthia, the genus Rhizobium, the genus Morganella, the genus Acinetobacter, the genus Pseudomonas, the genus Enterococcus, the genus Lactococcus, the genus Coprococcus, the genus Bacteroides, the genus Dorea, the genus Streptococcus, the genus Lachnospira, the genus Ruminococcus, the genus Corynebacterium, the genus Comamonas, the genus Gordonia, the genus Paraprevotella, the genus Mycobacterium, the genus Roseburia, the genus Slackia, the genus Escherichia, the genus Phascolarctobacterium, the genus Sutterella, the genus Virgibacillus, the genus Eggerthella, the genus Citrobacter, the genus Roseomonas, the genus Serratia, the genus Methanobrevibacter, and the genus Bilophila.

12. The method of claim 9, wherein the colon cancer in process (c) is diagnosed by comparing an increase or decrease in content of bacteria-derived extracellular vesicles between a colon polyp patient-derived sample and a colon cancer patient-derived sample through sequencing of a product of the PCR.

13. The method of claim 12, wherein the comparing comprises comparing an increase or decrease in content of:
extracellular vesicles derived from the bacteria belonging to the phylum Spirochaetes;
extracellular vesicles derived from one or more bacteria selected from the group consisting of the class Spirochaetes and the class Acidobacteria-6;
extracellular vesicles derived from one or more bacteria selected from the group consisting of the order Spirochaetales and the order Myxococcales;
extracellular vesicles derived from one or more bacteria selected from the group consisting of the family Spirochaetaceae and the family S24-7; or
extracellular vesicles derived from one or more bacteria selected from the group consisting of the genus Treponema, the genus Dialister, the genus Oscillospira, and the genus Eubacterium.

14. The method of claim 9, wherein the colon polyp in process (c) is diagnosed by comparing an increase or decrease in content of bacteria-derived extracellular vesicles between a normal individual-derived sample and a colon polyp patient-derived sample through sequencing of a product of the PCR.

15. The method of claim 14, wherein the comparing comprises comparing an increase or decrease in content of:
extracellular vesicles derived from one or more bacteria selected from the group consisting of the phylum Actinobacteria, the phylum Proteobacteria, and the phylum Euryarchaeota;
extracellular vesicles derived from one or more bacteria selected from the group consisting of the class Betaproteobacteria, the class Solibacteres, the class Gammaproteobacteria, the class Clostridia, the class Methanobacteria, and the class 4C0d-2;
extracellular vesicles derived from one or more bacteria selected from the group consisting of the order Burkholderiales, the order Sphingomonadales, the order Solibacterales, the order Stramenopiles, the order Pseudomonadales, the order Clostridiales, the order Oceanospirillales, the order Desulfovibrionales, and the order Methanobacteriales;
extracellular vesicles derived from one or more bacteria selected from the group consisting of the family Rhizobiaceae, the family Sphingomonadaceae, the family Exiguobacteraceae, the family Moraxellaceae, the family Pseudomonadaceae, the family Streptococcaceae, the family Peptostreptococcaceae, the family Comamonadaceae, the family Veillonellaceae, the family Bacteroidaceae, the family Paraprevotellaceae, the family Ruminococcaceae, the family Corynebacteriaceae, the family Christensenellaceae, the family Odoribacteraceae, the family Desulfovibrionaceae, the family Halomonadaceae, the family Alcaligenaceae, the family Barnesiellaceae, the family Methanobacteriaceae, and the family Rikenellaceae; or
extracellular vesicles derived from one or more bacteria selected from the group consisting of the genus Sphingomonas, the genus Alkanindiges, the genus Roseateles, the genus Rhizobium, the genus Morganella, the genus Proteus, the genus Exiguobacterium, the genus Acinetobacter, the genus Pseudomonas, the genus SMB53, the genus Lactococcus, the genus Coprococcus, the genus Streptococcus, the genus Bacteroides, the genus Ruminococcus, the genus Corynebacterium, the genus Odoribacter, the genus Clostridium, the genus Comamonas, the genus Paraprevotella, the genus Roseburia, the genus Citrobacter, the genus Klebsiella, the genus Virgibacillus, the genus Slackia, the genus Dialister, the genus Phascolarctobacterium, the genus Sutterella, the genus Halomonas, the genus Roseomonas, and the genus Methanobrevibacter.

16. The method of claim 9, wherein the subject sample is stool or urine.
